SEQUENCE LISTING (16 sequences)

SEQ ID NO: 1 - protein, 12 aa, Artificial Sequence
  Chemically synthesized
KRLEVDIDIKIR

SEQ ID NO: 2 - protein, 5 aa, Artificial Sequence
  Chemically synthesized
GARRP

SEQ ID NO: 3 - protein, 6 aa, Artificial Sequence
  Chemically synthesized
HHHHHH

SEQ ID NO: 4 - DNA, 38 bp, Artificial Sequence
  Primer tPA F-GST (1)
aaaagtcgacagccgccaccatggatgcaatgaagaga

SEQ ID NO: 5 - DNA, 33 bp, Artificial Sequence
  Primer tPA F-GST (2)
aaaagcggccgcccacttttgacaggcactgag

SEQ ID NO: 6 - DNA, 31 bp, Artificial Sequence
  Primer GST tag (3)
aaaagcggccgcctggctcctcttctgaatc

SEQ ID NO: 7 - DNA, 31 bp, Artificial Sequence
  Primer fibronectin F4-5-GST (4)
tgcaagatctatagctgagaagtgttttgat

SEQ ID NO: 8 - DNA, 32 bp, Artificial Sequence
  Primer fibronectin F4-5-GST (5)
tgatgcggccgccccacagaggtgtgcctctc

SEQ ID NO: 9 - DNA, 32 bp, Artificial Sequence
  Primer factor XII F-GST (6)
gaaacaagatctcagaaagagaagtgctttga

SEQ ID NO: 10 - DNA, 34 bp, Artificial Sequence
  Primer factor XII F-GST (7)
acgggcggccgcccggcctggctggccagccgct

SEQ ID NO: 11 - DNA, 31 bp, Artificial Sequence; identical to SEQ ID NO: 7
  Primer fibronectin F4-5-His (8)

SEQ ID NO: 12 - DNA, 50 bp, Artificial Sequence
  Primer fibronectin F4-5-His (9)
gggggcggccgctaatggtgatggtgatggtgcacagaggtgtgcctctc

SEQ ID NO: 13 - DNA, 28 bp, Artificial Sequence
  Primer GH-His-tPA F-His (10)
ggggagatcttaccaagtgatctgcaga

SEQ ID NO: 14 - DNA, 51 bp, Artificial Sequence
  Primer GH-His-tPA F-His (11)
gggggcggccgctaatggtgatggtgatggtgacttttgacaggcactgag

SEQ ID NO: 15 - DNA, 31 bp, Artificial Sequence; identical to SEQ ID NO: 7
  Primer GH-His-Fn F4-5-His (12)

SEQ ID NO: 16 - DNA, 50 bp, Artificial Sequence; identical to SEQ ID NO: 12
  Primer GH-His-Fn F4-5-His (13)

(19) United States
(12) Patent Application Publication
(10) Pub. No.: US 2006/0292683 A1
(43) Pub. Date: Dec. 28, 2006

(54) Cross-beta structures on microbial organisms

(75) Inventors: Martijn Frans Ben Gerard Gebbink, Eemnes (NL); Barend Bouma, Houten (NL); Herman Abel Bernard Wosten, Zeist (NL)

Correspondence Address: TRASK BRITT, P.O. BOX 2550, SALT LAKE CITY, UT, 84110, US

(73) Assignee: Crossbeta Biosciences B.V., Utrecht (NL)

(21) Appl. No.: 11/384,169

(22) Filed: Mar. 17, 2006

Related U.S. Application Data
(60) Provisional application No. 60/663,487, filed on Mar. 18, 2005.

(30) Foreign Application Priority Data
Mar. 18, 2005 (EP) 05075656.8

(51) Int. Cl. A61K 38/43; C12N 9/00; C12N 1/00

(52) U.S. Cl. 435/243; 424/94.1; 435/183

(57) ABSTRACT

The present invention discloses the use of a protease inhibitor in the preparation of a medicament for the treatment of microbial infections. The invention further discloses the use of a compound binding to a cross-β structure or an antibody specific for a cross-β structure in the preparation of a medicament for the treatment of microbial infections. The invention further discloses methods for attenuating microbial pathogens by deleting at least part of a gene encoding a cross-β structure forming protein. The invention also discloses an antimicrobial composition, and a kit for detecting microbial contamination in a solution or a substance.

### PRIORITY CLAIM

This application claims priority to U.S. Provisional Patent Application Ser. No. 60/663,487, filed Mar. 18, 2005, and European Patent Application Serial No. EP05075656.8, also filed Mar. 18, 2005, the contents of the entirety of each of which are incorporated by this reference.

### TECHNICAL FIELD

The invention relates to the field of biotechnology and microbiology, more specifically to antimicrobial medicines and antimicrobial immunogenic compositions.

### BACKGROUND

Amyloid fibrils have been associated with pathology in a class of degenerated diseases like, for example, Alzheimer's disease and Creutzfeldt-Jakob. Amyloid structures also occur on the surface of microbial organisms like fungi and bacteria. The proteins in amyloid fibrils, oligomers, deposits and aggregates in degenerative diseases like Alzheimer's disease and Creutzfeldt-Jakob differ from those on the surface of bacteria and fungi with respect to amino-acid sequence and peptide length.

The amyloid-like structures are generally called hydrophobins on fungi, chaplins on gram-positive bacteria, and curli or tafi or aggregative fimbriae on gram-negative bacteria.

Generally, the virulence of a microorganism depends on characteristics like, for example, colonization factors, exo- and endotoxin production, replication time, resistance to antibiotics, and survival in the host macrophages, just to name a few. Methods to attenuate microorganisms for vaccination purposes were generally directed to the above-mentioned characteristics. Antimicrobial medicines are generally antibiotic compounds or bacteriostatic compounds.

Since resistance of microorganisms to antibiotic and bacteriostatic compounds is an ever-increasing problem, new methods for combating microorganisms are needed.

### DISCLOSURE OF THE INVENTION

In certain aspects, the present invention provides at least one method or means that is suitable for at least in part combating a pathogenic microorganism or a microbial infection.

Disclosed herein is that microbial amyloid-like structures contain cross-β structure. Microbial hydrophobins, chaplins, tafis, curlis and aggregative fimbriae contain cross-β structure and are all capable of inducing protease activity in the body of a host. By inducing protease activity in a host tissue, a microorganism comprising cross-β, structure indirectly weakens the intercellular matrix thereby decreasing the integrity of the tissue. By this mechanism, the microorganism is able to invade the body.

Furthermore, the microbial cross-β structure is capable of binding intravascularly to factor XII. Binding of factor XII to the cross-β structure is followed by conversion of factor XII into a serine protease factor XIIa. The factor XIIa is involved in the release of bradykinin, which induces amongst other things, hemostasis and the release of more tissue type plasminogen activator (tPA) which is also a serine protease. Hence, microorganisms comprising cross-β structure, use the host cell's proteases to weaken the intracellular matrix of tissue and to cause intravascular hemostasis. Therefore, the presence of amyloid fibrils comprising cross-β structure renders a microorganism more virulent for a host.

The identification in the present invention of cross-β structure in the surface proteins of microorganisms now provides new methods for decreasing the virulence of a microorganism and offers new methods for inhibiting infection of a host by the microorganism.

A cross-β structure is a secondary/tertiary/quarternary structural element in peptides and proteins. A cross-β structure (also referred to as a “cross-beta” or a “crossbeta” structure or a “cbs”) is defined as a part of a protein or peptide, or a part of an assembly of peptides and/or proteins, which comprises an ordered group of β-strands, typically a group of β-strands arranged in a β-sheet, in particular a group of stacked β-sheets, also referred to as “amyloid.” A typical form of stacked β-sheets is in a fibril-like structure in which the β-sheets are stacked in either the direction of the axis of the fibril or perpendicular to the direction of the axis of the fibril. The direction of the stacking of the α-sheets in cross-β structures is perpendicular to the long fiber axis. A cross-β structure conformation is a signal that triggers a cascade of events that induces clearance and breakdown of the obsolete protein or peptide, a signaling pathway referred to as the “Cross-beta Pathway” for clearance of obsolete molecules and cells. When clearance is inadequate, unwanted proteins and/or peptides aggregate and form toxic structures ranging from soluble oligomers up to precipitating fibrils and amorphous plaques. Such cross-β structure conformation comprising aggregates underlie various diseases, such as, for instance, Huntington's disease, amyloidosis type disease, atherosclerosis, diabetes, bleeding, thrombosis, cancer, sepsis, inflammatory diseases, rheumatoid arthritis, transmissible spongiform encephalopathies such as Creutzfeldt-Jakob disease, Multiple Sclerosis, auto-immune diseases, diseases associated with loss of memory such as Alzheimer's disease, Parkinson's disease and other neuronal diseases (epilepsy), encephalopathy, encephalitis, cataract and systemic amyloidoses.

Cross-β structure is, for instance, formed during unfolding and refolding of proteins and peptides. Unfolding of peptides and proteins occur regularly within an organism. For instance, peptides and proteins often unfold and refold spontaneously during intracellular protein synthesis and/or during and/or at the end of their life cycle. Moreover, unfolding and/or refolding is induced by environmental factors such as, for instance, pH, glycation, oxidative stress, heat, irradiation, mechanical stress, proteolysis and so on. The terms unfolding, refolding and misfolding relate to the three-dimensional structure of a protein or peptide. Unfolding means that a protein or peptide loses at least part of its three-dimensional structure. The term refolding relates to the coiling back into some kind of three-dimensional structure. By refolding, a protein or peptide can regain its native configuration, or an incorrect refolding can occur. The term “incorrect refolding” refers to a situation when a three-dimensional structure other than a native configuration is formed. Incorrect refolding is also called misfolding. Unfolding and refolding of proteins and peptides involve the risk of cross-β structure formation. Formation of cross-β structure sometimes also occurs directly after protein synthesis, without a correctly folded protein intermediate.

Also disclosed is that a protease inhibitor is capable of decreasing the serine protease activity caused by cross-β structure. Therefore, the dissolving action on tissue and on blood clots is decreased, and the invasion of tissue by the microbial organism is at least in part decreased. Administration of the protease inhibitor as a medicine is very useful for treatment of an animal and/or a human being suffering from an infection with a microbial organism. Therefore, the present invention discloses the use of a protease inhibitor in the preparation of a medicament for the treatment of microbial infections. Preferably, the protease inhibitor is specific, i.e., the inhibitor essentially only inhibits, for example, serine protease factor XII.

A compound capable of binding to a cross-β structure also prevents the increase in protease activity and is, therefore, an inhibitor of a microbial infection. Therefore, the present invention also includes the use of a cross-β structure binding compound, like, for example, selected from those listed in Tables 3 through 5, in the preparation of a medicament for the treatment of microbial infections. Preferably, the compound comprises an antibody, or a functional fragment or derivative thereof, and/or a finger domain of a protease, specific for cross-β structure. An example of such a finger domain is described in Example 2 and in FIG. 2. Most preferably, the antibody comprises a bi-specific antibody capable of recognizing and binding to cross-β structure.

Modern technology has enabled a skilled person to produce and select monoclonal antibodies. In this development also bi-specific antibodies are constructed. Generally a bi-specific antibody comprises two different binding specificities in single molecule. In, for example, a bi-specific antibody against cross-β structure of a microorganism, one antibody specificity is directed against the cross-β structure, while the other antibody specificity is preferably directed against another antigen of the microorganism. A bi-specific antibody has the advantage that the binding rate is increased because the antibody binds to more than one antigen. In one embodiment, a bi-specific antibody is generated which is directed against a particular (pathogenic) strain of microorganisms while leaving other (non-pathogenic) strains of the same microorganism untouched. Therefore, the present invention discloses the use according to the invention, wherein the antibody is a bi-specific antibody against a cross-62 structure and a microbial antigen.

In yet another preferred embodiment, the cross-β structure binding compound is any of the compounds mentioned in Tables 3 or 4 or 5 and, hence, the invention also discloses the use of a cross-β structure binding compound in the preparation of a medicament for the treatment of microbial infections, wherein the cross-β structure binding compound is selected from Tables 3 or 4 or 5.

It is clear to the skilled person that also combinations of any of the mentioned cross-β structure binding compounds are suitable for the treatment of microbial infections.

The above-described use of a protease inhibitor and/or cross-β structure binding compound (for example, an antibody and/or a bi-specific antibody or any of the compounds mentioned in Tables 3 or 4 or 5) is useful for the treatment of infections with microorganisms. Both gram -negative and gram-positive bacteria and also fungi have protein comprising cross-β structure on the surface. Therefore, the present invention discloses a use according to the invention, wherein the microorganism is cross-β structure-comprising Gram-positive microorganism. Because cross-β structure on a Gram-positive microorganism is generally present in a specific structure like a hydrophobin, or chaplin, the present invention discloses the use according to the invention, wherein cross-β structure comprises a hydrophobin or a chaplin.

Because a hydrophobin generally occurs on a fungus, the present invention discloses the use according to the invention, wherein the microorganism is a fungus. Hydrophobins have been detected in both the Ascomycetes and the Basidiomycetes. Both phyla represent most species in the fungal kingdom. Hydrophobins are generally expressed in dimorphic fungi. For the present invention, treatment of infections with pathogenic fungi is of preferred interest.

Because a chaplin generally occurs on a Gram-positive bacterium, the present invention discloses a use according to the invention, wherein the microorganism is a Gram-positive bacterium. In Gram-positive bacteria, chaplins are generally found on Streptomyces species and in other actinomycetes. Therefore, the invention provides a use, wherein the Gram-positive bacterium is an actinomycete or a streptomycete bacterium. Yet another example of a Gram-positive bacterium is a Staphylococcus, for example, a Staphylococcus aureus.

Because a curli or a tafi or a thin aggregative fimbria generally occurs on Gram-negative bacteria, the present invention discloses a use according to the invention, wherein the microorganism is a Gram-negative bacterium.

Important pathogens covered with curli or tafi or thin aggregative fimbriae are bacteria from the genera E. coli and Salmonella. Therefore, the present invention discloses a use according to the invention, wherein the Gram-negative bacterium is an E. coli bacterium or a Salmonella bacterium.

By disclosing the function of cross-β structure in the amyloid fibril for the virulence of a microorganism, the present invention discloses a method for decreasing the virulence of a microorganism by at least in part preventing the formation of cross-β structures. Decreasing is preferably achieved by deleting at least a part of a gene encoding an amyloid fibril forming protein. This results in at least partly decreased amyloid fiber formation on the surface and, therefore, decreased cross-β structure presence. The microorganism with decreased virulence is safer for a host and is useful as an immunogenic composition. Therefore, the present invention discloses a method for producing a less virulent microorganism, comprising deleting at least a part of a gene of the microorganism encoding an amyloid fibril forming protein. It is however also possible to make other mutations that result in less cross-β structure on the exterior of a microorganism. Such mutations include but are not limited to insertions, replacements etc.

Because part of the amyloid fibril is formed by cross-β structure, decreasing the amount of a cross-β structure-forming protein is a suitable way of decreasing the virulence of a microorganism without deleting other antigens. Such a microorganism preferably is more suitable for use in an immunogenic composition than a microorganism from which antigens have been stripped or altered in a process of attenuation. Therefore, the present invention discloses an immunogenic composition comprising a microorganism from which at least one gene, encoding a cross-β structure forming protein, has been at least in part deleted (or mutated in any other way, such that the amount of cross-β structure is at least in part decreased).

In a preferred embodiment of the invention, a protease inhibitor of the invention is used in combination with a fungicide compound. The fungicide preferably potentiates the effect of the protease inhibitor in decreasing an infection with a pathogenic fungus. A fungicide preferably is a compound generally known for its fungicidal activity. In this invention, for application to a host, (such as an animal or a human being), use of fungicidal compounds is restricted to those fungicides that are suitable for use on or in the host. Therefore, the present invention discloses a composition comprising a protease inhibitor and a fungicide.

In another preferred embodiment, a protease inhibitor of the invention is used in combination with a bactericide compound. The bactericide preferably potentiates the effect of the protease inhibitor in decreasing an infection with a pathogenic bacterium. In this invention, for application to a host (such as an animal or a human being), use of bactericidal compounds is restricted to those bactericides that are permitted for use on or in the host. Therefore, in a preferred embodiment, the present invention discloses a composition comprising a protease inhibitor and a bactericide.

Because cross-β structure induces serine proteases, inhibition of the serine proteases by specific serine protease inhibitors is preferred. Therefore, the present invention discloses a composition according to the invention, wherein the protease inhibitor is a serine protease inhibitor.

The invention further provides a composition comprising a protease inhibitor and a cross-β structure-binding compound. Such composition is particularly suitable for counteracting the microbial cross-β structure activity. The cross-β structure-binding compound preferably comprises an antibody, or a functional fragment or derivative thereof, and or a finger domain of a protease, specific for cross-β structure. In order to specifically counteract bacteria and/or fungi, the composition preferably further comprises a bactericide and/or a fungicide.

In yet another embodiment, the invention provides a (pharmaceutical) composition comprising a cross-β structure binding compound. In a preferred embodiment, the cross-β structure binding compound is selected from Tables 3 or 4 or 5. In yet an even more preferred embodiment, the compositions comprises at least two different cross-β structure binding compounds, which are preferably selected from Tables 3 or 4 or 5. In yet an even more preferred embodiment, the compositions comprise at least one or more of the cross-β structure binding compounds chaperones, soluble CD36 or soluble RAGE. In another embodiment, the composition further comprises at least one fungicide or at least a bactericide or a combination thereof.

In another embodiment, the invention provides a kit for detecting microbial contamination of a solution and/or a substance, the kit comprising a cross-β structure binding compound and a means for detecting binding of the cross-β structure to the binding compound. The means for detecting binding of cross-β structure to a cross-β structure-binding compound preferably comprises a tPA- and/or factor XII activation assay. In another preferred embodiment, the means for detecting binding of cross-β structure to a cross-β structure binding compound comprises visualization of a staining compound.

In yet another embodiment, the invention provide a method for detecting the presence of a microorganism in a solution and/or a substance, the method comprising providing a cross-β structure binding compound to the solution and/or substance and detecting whether any bound complex is present.

Examples of a suitable cross-β structure binding compound are, for example, outlined in Tables 3 or 4 or 5, and include a non-proteinaceous molecule, for example, a dye (Congo red or Thioflavin).

In a preferred embodiment, a cross-β structure binding compound is attached to a (solid) support or phase (i.e., is immobilized) such as, for example, on a sphere or a particle or a bead or a sheet or a strand of latex or agarose or glass or plastic or metal or any other suitable substance for immobilization of molecules. Such immobilization is especially useful when bound and unbound proteins must be separated. Depletion of a fluid from cross-beta structure and/or a protein comprising a cross-beta structure can be assessed, and/or enrichment of a solid support with bound cross-β structure binding compound with cross-beta structure and/or a protein comprising a cross-beta structure can be assessed, after contacting a fluid to a cross-β structure binding compound that is immobilized on a solid support. For example, a spike of a reference cross-beta structure can be applied to a tester sample and a control or reference sample. When contacting the samples with a cross-β structure binding compound the amount of cross-beta structure originally present in the sample will determine the amount of reference cross-beta structure that will bind to the cross-β structure binding compound. The differences in amount of reference cross-beta structure in a control sample and in a tester sample after contacting both samples to the cross-β structure binding compound can be assessed, for example, with a(n) (sandwich) ELISA specific for the reference cross-beta structure or, for example, by fluorescence measurement when a fluorescent label is coupled to the reference cross-beta structure. Alternatively, the amount of reference cross-beta structure bound to the cross-β structure binding compound can be assessed similarly. In yet an alternative approach, all proteins in a tester sample and in a reference or control sample can be labeled, for example, with biotin or a fluorescent label, prior to exposure to a cross-beta structure binding compound. The amount of labeled protein comprising cross-beta structure bound to the cross-β structure binding compound can subsequently be quantified and compared. In yet another approach, cross-beta structure that is bound to cross-β structure binding compound after contacting a reference sample and a tester sample with the cross-β structure binding compound, can be quantified after elution from the cross-β structure binding compound immobilized on a solid support, using a chromogenic assay. In the chromogenic assay, for example, dilution series of eluates of cross-β structure binding compound ligands are mixed with tissue-type plasminogen activator, plasminogen, a chromogenic substrate for plasmin and a suitable reaction buffer, and conversion of the substrate is followed in time upon 37° C. incubation.

Now that we have disclosed that a large number of microorganisms display cross-β structure on their exterior, this information is used to upgrade any solution, for example, a solution meant for human use. In one of the embodiments, the to be given solution is allowed to flow through a column in which matrix material is provided with immobilized cross-β structure binding compound (for example, one of the compounds as outlined in Tables 3 or 4 or 5). Any microorganism that is present in the solution and that comprises cross-β structure on its exterior will bind to the column and the solution is thus (at least in part) cleared from the microorganisms. Preferably, the conditions are such (for example, due to the presence of enough cross-β structure binding compound) that all microorganisms that comprise cross-β structure on their exterior are removed from the solution. In yet another embodiment, a cross-β structure binding compound is added to a solution and by binding of the binding compound to cross-β structure present on the exterior of a microorganism, the microorganism is subsequently not effective in provoking an adverse or unwanted or excessive reaction in a mammal that is treated with the solution. It is clear that in this embodiment, the cross-β structure binding compound may not be harmful to the mammal which subsequently receives the thus-treated solution. The invention thus also provides a method for at least in part removing a microorganism from a solution comprising contacting the solution with a cross-β structure binding compound. Preferably, the solution is subsequently used in the treatment of a mammal (preferably a human being). An example of such a solution is a dialysis solution.

The experimental part discloses that a cross-β structure binding compound is very useful in at least in part decreasing the pathogenicity of a microorganism and, hence, in yet another embodiment, the invention provides a method for decreasing the pathogenicity of a microorganism comprising providing the microorganism with a cross-β structure binding compound. With such a method cross-β structure present on the exterior of a microorganism is blocked or covered or shielded or neutralized and cannot participate in invading a mammal. Preferably, the microorganism is an E. coli or a Staphylococcus. The invention also provides the use of a cross-β structure binding compound for reducing the pathogenicity of a microorganism. Examples of suitable binding compounds are Thioflavin T (ThT), Congo red, intravenous immunoglobulins (IgIV), tissue-type plasminogen activator (tPA) or any of the other mentioned cross-β structure binding compounds of Tables 3 or 4 or 5.

The experimental part further discloses that interaction between an antigen-presenting cell and a pathogen is influenced by the presence or absence of a cross-β structure binding compound and, hence, the invention also provides a method for modifying the interaction between an antigen-presenting cell and a microorganism comprising changing the amount of a cross-β structure binding compound. It is clear that the microorganism is a microorganism that comprises cross-β structure on its exterior (for example, an E. coli or a Staphylococcus). Whether the amount of cross-β structure binding compound is increased or decreased depends on the specific situation. If one, for example, wants to stimulate the contact between a microorganism and an antigen-presenting cell, the amount of cross-β structure binding compound is reduced and if one wants to (at least in part) inhibit the contact between a microorganism and an antigen-presenting cell, the amount of cross-β structure binding compound is increased. An example in which one would like to increase the amount of cross-β structure binding compounds, is in patients with deleterious autoimmune disease, like, for example, multiple sclerosis (MS) or rheumatoid arthritis. These diseases are typically accompanied with all kinds of infection. By at least in part decreasing the interaction between a microorganism involved in the infection and an antigen-presenting cell, the human being suffering from MS or rheumatoid arthritis does not have to cope with the consequences of a microbial infection. In a preferred embodiment, the antigen-presenting cell is a dendritic cell.

The present invention refers at multiple locations to a microbial infection. It is clear to the skilled person that such different microbial infections may lead to a multitude of diseases. Examples of diseases that are preferably treated or prevented (i.e., prophylactic treatment) are mastitis or sepsis or any of the pathological conditions and diseases related to infection with any of the pathogens summarized in Table 6.

The invention further provides a method for in vitro determining whether a compound is capable of decreasing the pathogenicity of a microorganism comprising any of the methods as outlined in the experimental part herein. With such a method the efficiency of multiple cross-β structure binding compounds is easily determined and compared.

In yet another embodiment, the invention further provides one of the following methods:

(i) A method for at least in part inhibiting maturation of an antigen-presenting cell by a microorganism, comprising providing to the antigen-presenting cell and/or to the microorganism a cross-β structure binding compound.

In a preferred embodiment, the antigen-presenting cell is a dendritic cell.

(ii) A method for at least in part inhibiting induction of platelet activation by a microorganism, comprising providing to the platelet and/or to the microorganism a cross-β structure binding compound.

(iii) A method for at least in part inhibiting invasion of a microorganism into a host cell by extra-cellular matrix breakdown, comprising providing to the microorganism and/or the host cell a cross-β structure binding compound.

(iv) A method for at least in part reducing the vitality of a microorganism comprising providing to the microorganism a cross-β structure binding compound.

In a preferred embodiment, the microorganism is Gram-positive or a Gram-negative bacterium. In yet another preferred embodiment, the microorganism is a Streptomyces species or a Staphylococcus species or a Salmonella species or an E. coli or any of the pathogens mentioned in Table 6.

In another preferred embodiment, the microorganism is involved in the induction of sepsis and hence, the method is especially useful in the treatment of sepsis.

Any of the mentioned methods may be performed in vitro as well as in vivo and, hence, the invention also provides use of a cross-β structure binding compound in the preparation of a medicament in the treatment of a microbial infection.

### BRIEF DESCRIPTION OF THE DRAWINGS

FIG. 1: Recombinant finger constructs and Streptomyces coelicolor bacteria activate the fibrinolytic system in vitro, whereas bacterium cells of the Δrdlab mutant strain, which lacks the amyloid-like core chaplins, does not. FIG. 1A: Recombinant human fibronectin type I or finger domains. FIG. 1B: Cells of the bacterium Streptomyces coelicolor wild-type strain (wild-type) are able to activate the tPA-mediated conversion of zymogen plasminogen to the active serine protease plasmin, as determined in a chromogenic assay with the plasmin substrate S-2251. In contrast, cells of a mutant strain Δrdlab, which lack the gene encoding for the chaplins core proteins (mutant), that has amyloid-like conformation, do not activate plasmin generation. Positive control was 100 μg/ml amyloid γ-globulins (positive control), negative control was buffer.

FIG. 2: Binding of tPA, factor XII, fibronectin and finger domains thereof to compounds with cross-β structure. Panels A-C: Full-length purified tPA (Panel A), factor XII (Panel B) and fibronectin (Panel C) bind to immobilized peptides with cross-β structure conformation, in an ELISA. Panels D-F: In an ELISA, the recombinant Fibronectin type I, or finger domains (Panel F) of tPA (Panel D), factor XII (Panel E) and fibronectin (Panel F) bind specifically to immobilized amyloid-like peptides with cross-β structure conformation. The control free GST tag does not bind. Panel G: In an ELISA, immobilized chemically synthesized fibronectin type I domain of tPA (tPAFbiotin) specifically captures glycated hemoglobin (Hb-AGE) from solution, and not control hemoglobin. Panel H: In an ELISA, recombinantly expressed fibronectin type I domains 4-5 of fibronectin, N-terminally tagged to growth hormone and a His₆-tag, and C-terminally tagged to a His₆-tag (FnF4,5his), specifically captures Hb-AGE from solution, and not control hemoglobin.

FIG. 3: Reactive oxygen species production by microvascular bEnd.3 endothelial cells upon exposure to Escherichia coli TOP10 cells is reversed by pre-incubation of the pathogen with cross-beta structure binding compounds. FIG. 3A: Binding of cross-beta structure binding compound IgIV to immobilized glycated hemoglobin comprising cross-beta structure in an ELISA set-up. FIG. 3B: ROS production by bEnd.3 ECs upon exposure to 1/120^{th} volume PBS, 100 μM H₂O₂, 6×10⁸ E. coli TOP10 cells/ml or 6×10⁸ E. coli cells/ml that were pre-incubated with cross-beta structure binding compounds Congo red, ThT, tPA and IgIV (“cbs binders”). Up-regulation of ROS by the ECs was determined by measuring fluorescence of a probe for ROS. PBS was used as a negative control, 100 μM H₂O₂ was used as a positive control for ROS induction.

FIG. 4: Activation of factor XII by Escherichia coli strain TOP10 is inhibited by cross-beta structure binding compounds, and reactive oxygen species production by bEnd.3 endothelial cells is inhibited by cross-beta structure binding compounds. FIG. 4A: Exposure of cultured murine brain microvascular endothelial cells (bEnd.3) to E. coli Top10 induces up-regulation of reactive oxygen species (ROS), as measured in a kinetic fluorescent assay. Serial pre-incubation of the E. coli cells with cross-beta structure binders Thioflavin T, Congo red and a mixture of tissue-type plasminogen activator and intravenous immunoglobulins (“cbs binders”) lowers ROS levels induced by E. coli. PBS was used as a negative control, 100 μM H₂O₂ was used as a positive control for ROS induction. FIG. 4B: Escherichia coli TOP10 (E. coli) induces factor XII activity in a chromogenic factor XII/prekallikrein activation assay. Serial pre-incubation of the E. coli cells with cross-beta structure binders Thioflavin T, Congo red and a mixture of tissue-type plasminogen activator and intravenous immunoglobulins (“cbs binders”) diminish factor XII activation by E. coli to background levels observed with buffer only. Kaolin at 150 μg/ml was used as a positive control. Including factor XII in the experiments was essential; discarding factor XII did not result in any conversion of the chromogenic kallikrein substrate (not shown).

FIG. 5: Staphylococcus aureus Newman and Escherichia coli TOP10 induce platelet aggregation, which is inhibited by cross-beta structure binding compounds.

FIG. 5A: Platelets in platelet rich plasma (PRP) from healthy human donor A readily aggregate upon stimulation by Staphylococcus aureus Newman (S. aureus). Pretreatment of the S. aureus cells with cross-beta structure binding compounds Thioflavin T, Congo red and a mixture of tPA and IgIV (S. aureus+cbs binders) inhibits S. aureus induced platelet aggregation. FIG. 5B: Similar experiment as in FIG. 5A. Now, S. aureus cells were used after 24 hours storage at 4° C., and blood was donated by anonymous donor B. Again, S. aureus readily induces platelet aggregation, which is in part reversed upon pre-treatment of S. aureus with cross-beta structure binding compounds. FIG. 5C: Platelets aggregate when exposed to Escherichia coli TOP10 cells. Cross-beta structure binding molecules reverse this platelet activating properties. FIG. 5D: Positive and negative controls TRAP and buffer. The graph shows that TRAP only activate platelets when present at a cut-off level of over 2 μM.

FIG. 6: Vitality of S. aureus Newman bacteria and E. coli TOP10 bacteria before and after incubation with cross-beta structure binding compounds. Bacterium cultures of 1 ml were grown for six hours at 37° C. with vigorous shaking and aeration, by inoculating 1 ml LB medium with 25 μl cell suspension. After six hours, cell density was measured by an absorbance reading at 600 nm with 50× diluted cell suspension. Vitality of E. coli or S. aureus incubated with PBS was compared with vitality of the bacteria after incubations with cross-beta structure binding compounds Thioflavin T, Congo red and a mixture of tPA and IgIV.

FIG. 7: Binding of tPA to E. coli TOP10 and S. aureus Newman, assessed with a tPA-plasminogen activation assay. Binding of tPA to E. coli TOP10 and S. aureus Newman was determined using a tPA/plasminogen activation assay including a chromogenic plasmin substrate. The bacteria were pre-incubated with buffer (E. coli, S. aureus) or with cross-beta structure binding compounds (“cbs binders”); first 2.5 mM Thioflavin T, then 5 mM Congo red and finally 25 μM tPA+25 mg/ml IgIV (E. coli+cbs binders, S. aureus+cbs binders). In the assay 1.3×10⁸ E. coli cells/ml and 1.8×10⁸ S. aureus cells/ml are used. tPA is omitted in the reaction mixture and, therefore, plasmin generation can only occur when an external source of tPA is introduced in the assay.

FIG. 8: Activation of the contact system of coagulation by E. coli MC4100 either or not exposing amyloid cross-beta structure comprising core protein curli. Exposing factor XII, prekallikrein, high molecular weight kininogen and chromogenic kallikrein substrate Chromozym-PK to E. coli MC4100 grown for 44 hours at 26° C. (+curli) results in more potent activation of the contact system of coagulation than exposure to E. coli MC4100 grown for 24 hours at 37° C. (−curli). Negative control: 1 mg/ml bovine serum albumin, positive control: 1 mg/ml bovine serum albumin+150 μg/ml kaolin.

FIG. 9: Production of ROS by bEnd.3 ECs is influenced by E. coli and S. aureus pre-incubated with cross-beta structure binding compounds. Panel A: ROS production was followed in time using fluorescent probe CM-H₂DCFDA. As positive control, overnight cultured ECs (64×10³ cells/well) were exposed to a concentration series of H₂O₂. Panel B: Influence of ROS production upon exposure of ECs (128×10³ cells/well, cultured overnight) to E. coli TOP10 cells and to bacteria that were pre-incubated with cross-beta structure binding compounds Thioflavin T, Congo red, tPA, IgIV is shown, as well as of ECs that were co-incubated with E. coli and indicated cross-beta structure binding compounds. Panel C: As in Panel B, with S. aureus Newman cells.

FIG. 10: Influence of pathogens on coagulation. Role of cross-beta structure binding compounds. Panel A: In a PT set-up, coagulation of human plasma was determined after pre-incubating the plasma with buffer or with 6.5×10⁹ E. coli TOP10 cells/ml. The E. coli were pre-incubated with PBS (“buffer”) or with cross-beta structure binding compounds ThT, Congo red, tPA, IgIV (“cbs binders”). Panel B: In an aPTT set-up the same samples were analyzed as in Panel A.

FIG. 11: Maturation of human immature dendritic cells by pathogens is influenced by cross-beta structure binding compounds. FIG. 11A: CD40 cell surface expression on DCs that were stimulated with buffer-treated E. coli TOP10 (E. coli) or cross-beta structure binding compound-incubated E. coli (E. coli+cbs binders). FIG. 11B: As in FIG. 11A, for DCs that were similarly stimulated with S. aureus Newman and S. aureus Newman that were pre-incubated with cross-beta structure binding compounds Thioflavin T, Congo red, tissue-type plasminogen activator and intravenous immunoglobulins. FIGS. 11C-11D: Cell surface expression of endocytic receptor CD36 upon stimulation with E. coli or E. coli pre-incubated with cross-beta structure binders (FIG. 11C), or with S. aureus or S. aureus pre-incubated with cross-beta structure binding compounds (FIG. 11D). FIGS. 11E-11F: Cell surface expression of endocytic receptor CD206 upon stimulation with E. coli or E. coli pre-incubated with cross-beta structure binders (FIG. 11E), or with S. aureus or S. aureus pre-incubated with cross-beta structure binding compounds (FIG. 11F). The negative control was non-stimulated DCs (“−control”). Mean fluorescence intensity (MFI) ratios are shown for all studied cell surface markers: MFI signal of specific markers (CD40, CD36, or CD206)/MFI signal of background (signal to noise ratio).

FIG. 12: Tissue factor up regulation in THP-1 monocytes upon stimulation with S. aureus is blocked by cross-beta structure binding compounds. FIG. 12A: S. aureus induces up-regulation of TF synthesis in THP-1 monocytes, as assessed with a chromogenic activated factor X assay in the presence of substrate S2765 and factor VII. Upon pre-incubation of S. aureus with cross-beta structure binding compounds ThT, Congo red, tPA and IgIV, TF expression returns to basal levels observed with negative control (buffer incubated THP-1). S. aureus cells alone did not induce factor X activity. FIG. 12B: Glycated hemoglobin (Hb-AGE) and amyloid-β (Aβ) are potent inducers of TF up-regulation in THP-1 monocytes. Negative controls: buffer, freshly dissolved Aβ, freshly dissolved Hb, positive control: 10 μg/ml LPS.

FIG. 13: Summary of the pathogenic nature of E. coli and S. aureus as measured in the described bioassays, and the influence of cross-beta structure binding compounds on pathogenicity. Pathogenicity of E. coli and S. aureus can have several faces. The measured influence of the pathogens on representative cells and enzymes that can contact pathogens during invasion of a host, in various bioassays, as described in the current examples, is combined. In the figure open arrows indicate a stimulatory activity of the pathogens, whereas blocking arrows indicate the inhibiting nature of cross-beta structure binding compounds. In FIG. 13A: A summary is given of all data gathered with Gram-negative bacterium E. coli, in FIG. 13B: The summary is given for Gram-positive bacterium S. aureus. (1) Maturation of dendritic cells (DC) by the pathogens is efficiently inhibited by cross-beta structure binding compounds (“cbs binder”). This shows that cross-beta structure on the surface of the pathogens aids in modulation of the immune response. (2) The pathogens induce platelet activation resulting in aggregation. The pro-thrombotic nature of the pathogens is in part reversed by cross-beta structure binding compounds. (3) The pathogens bind tPA efficiently, readily resulting in plasmin formation, which contributes to invasion of the pathogens by extra-cellular matrix breakdown. (4) Exposure of endothelial cells (EC) to the pathogens result in impaired ECs as indicated by up regulation of reactive oxygen species (ROS). (5) Vitality of the pathogens is lowered upon incubation of the bacteria with cross-beta structure binding compounds. Inhibiting proliferation of pathogens may be beneficial during anti-microbial treatment regimes for infected patients. (6) E. coli triggers the key enzyme of the intrinsic pathway of coagulation, factor XII, resulting in a pro-coagulant state. Triggering the coagulation cascade is inhibited by pre-incubating the E. coli with cross-beta structure binding compounds. (7) S. aureus exposure to monocytes results in a pro-coagulant state due to the up regulation of tissue factor (TF). Pre-incubation of the S. aureus with cross-beta structure binding compounds reduced TF up regulation back to levels found with negative control (buffer). Triggering of platelet aggregation (2), fibrinolytic activity (3), EC activation (4), enzymes involved in coagulation cascades (6) and TF expression (7) altogether can contribute to the onset of disseminated intravascular coagulation (DIC), a syndrome seen often during sepsis.

### DETAILED DESCRIPTION OF THE INVENTION

The invention is further described with the use of the following illustrative examples.

### EXAMPLE 1

Bacterial Cells with Amyloid-Like Core Protein Activate the Fibrinolytic System in Vitro

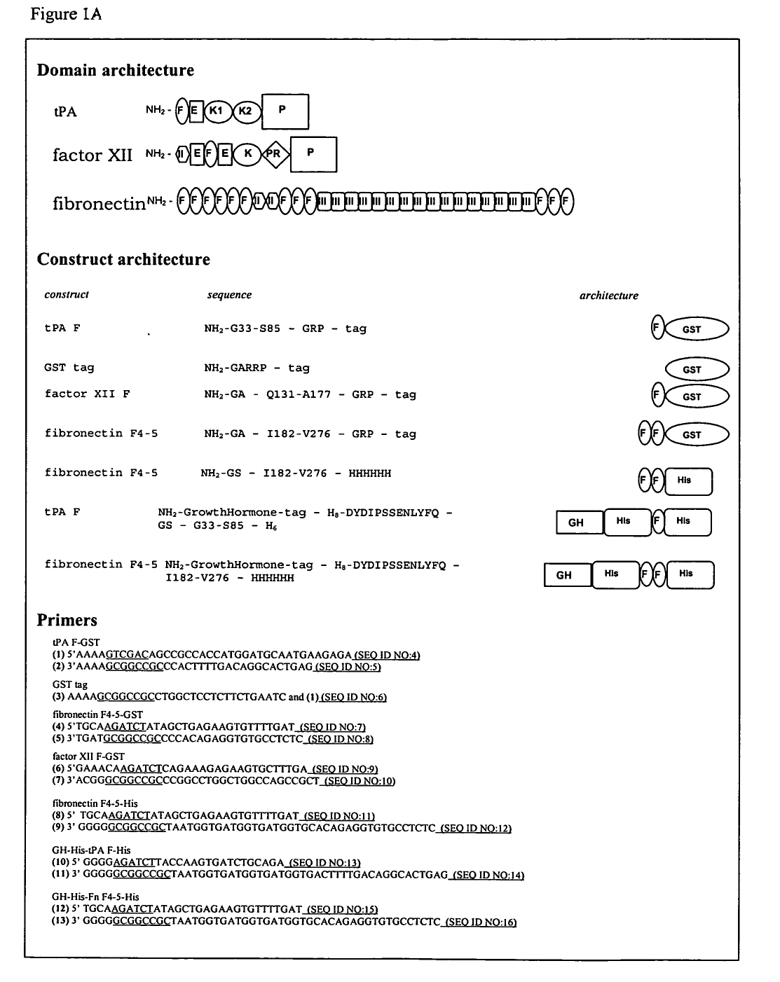

The Streptomyces coelicolor bacterium strain comprises a family of core proteins, chaplins A-H, which have adopted amyloid-like fibril conformation (Claessen et al., 2003). We now show that contacting the wild-type strain with tPA, plasminogen and plasmin substrate S-2251 results in activation of tPA and plasminogen (FIG. 1B). Interestingly, a mutant strain that lacks the amyloid-like core protein, does not stimulate tPA activation (FIG. 1B).

The data show that the presence of the chaplin core proteins with amyloid-like conformation, on the surface of Streptomyces coelicolor cells activates the fibrinolytic pathway, by activation of tPA. Cells of a mutant strain lacking the genes that encode for the amyloid-like chaplin do not induce tPA activation. Activation of the fibrinolytic cascade or of factor XII, the key protein in the contact system of blood coagulation, by several different pathogens shows that in general core proteins are involved in interactions with the host haemostatic system. Our determination now of the F domain as the specific domain that interacts with protein aggregates (see Example 2) comprising amyloid-like cross-β structure conformation enables for tracing of pathogens with exposed amyloid-like proteins on their surface. The tPA- and factor XII activation assays are indicative in quality measures of solutions suspected for the presence of pathogenic infections.

### EXAMPLE 2

tPA, Factor XII, Fibronectin and the Fibronectin Type I Domains of tPA, Factor XII and Fibronectin Bind to Protein Aggregates with Cross-β Structure Conformation

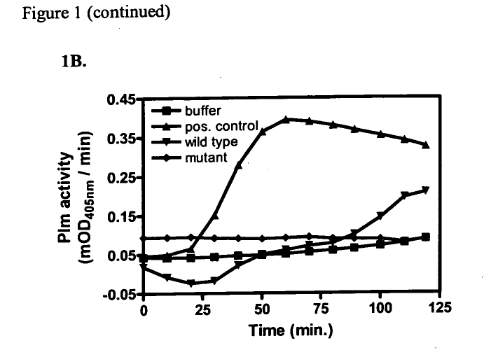

Previously, we established that tissue-type plasminogen activator specifically interacts with protein and peptide aggregates that comprise a cross-β structure conformation, a structural element found in amyloid-like polypeptide assemblies (Bouma et al., 2003; Kranenburg et al., 2002). Now, we expanded this analysis to other proteins that resemble tPA domain architecture and we separated domains of tPA. Binding of full-length tPA, factor XII and fibronectin, as well as of fibronectin type I (finger, F) domains of tPA and factor XII and F4-5 of fibronectin, to protein and peptide aggregates with cross-β structure conformation was analyzed in an ELISA. In FIG. 1 it is shown that the full-length proteins as well as the recombinant F domains bind specifically to cross-β structure rich compounds. Binding of tPA and factor XII was established for immobilized amyloid-β(1-40) (Aβ) with amyloid-like properties, fibrin peptide FP13, that encompasses the tPA activating sequence 148KRLEVDIDIKIR160 (SEQ ID NO:_{——————}) of the fibrin α-chain, TTR11, which is an 11 amino-acid residues peptide from transthyretin that forms cross-β structure, and LAM12, which is a 12 amino-acid residues peptide from laminin that forms cross-β structure (FIG. 2, Panels A and B). Negative controls were freshly dissolved, monomerized Aβ and non-amyloid murine islet amyloid polypeptide (mIAPP). For fibronectin, human amyloid IAPP is depicted instead of LAM12 (FIG. 2, Panel C). The separate F domains also bind to aggregates with cross-β structure, as depicted for Aβ and tPA F in FIG. 2, Panel D, and for all aggregates and factor XII F and fibronectin F4-5 in FIG. 2, Panels E and F. In addition, immobilized tPA F with a biotin tag and fibronectin F4-5 with a His-tag specifically capture glycated hemoglobin with amyloid-like properties in solution (FIG. 2, Panels G and H).

### Material and Methods

Cloning and Expression of Recombinant Fibronectin Type I Domains

Amino-acid sequences of recombinantly produced domains of tPA, fibronectin and factor XII, and the domain architecture of the recombinant constructs are depicted in FIG. 1A. Amino-acid residue numbering is according to SwissProt entries. Each construct has a carboxy terminal GST-tag (GST). Factor XII fibronectin type I domain (F) and fibronectin F4-5 are preceded by two amino acids (GA), following the C-terminus of the tPA propeptide. All F constructs are followed by the (G)RP sequence derived from the original pMT2-GST vector. For each recombinant construct, the oligonucleotides that were used for PCR are listed in FIG. 1A. The relevant restriction sites are underlined. The tPA fibronectin type I domain (F, finger domain), together with the tPA propeptide, was amplified using 1 ng vector Zpl7 containing tPA and oligonucleotides 1 and 2, digested with SalI and NotI and cloned into pMT2SM-GST. As a result Schistosoma japonicum glutathion-5-transferase (GST) is fused to the C-terminus of the expressed constructs. The constructs were subsequently ligated with SalI and EcoRI in pGEM3Zf(−) (Promega, Madison, Wis., USA). The resulting plasmid was used as a cloning cassette for preparation of factor XII F and fibronectin F4-5 constructs. The selection of fibronectin type I domains of fibronectin was based on the following reasoning. tPA binds to fibrin with its fibronectin type I domain and competes with fibronectin for fibrin binding. A fibrin binding-site of fibronectin is enclosed in its fibronectin type I4-5. We show here that the fibronectin type I domain of tPA mediates binding to amyloid. This suggests that also the fibrin-binding fibronectin type I domains of fibronectin can bind to amyloid. All domains were cloned after the tPA propeptide using a BglII restriction site that is present between the tPA propeptide region and the F domain, and the NotI or KpnI site that is present in front of the thrombin cleavage site. Subsequently, constructs were ligated HindIII and EcoRI in the pcDNA3.1 expression vector (Invitrogen, The Netherlands). This results in, e.g., pcDNA3.1-factor XII F-GST and pcDNA3.1-Fn F4-5-GST. In addition, the GST tag alone, preceded by the tPA propeptide, was cloned into pcDNA3.1. The separate GST-tag has five additional residues at the N-terminus (GARRP). tPA cDNA was a kind gift of M. Johannessen (NOVO Research Institute, Bagsvaerd, Denmark). The cDNA encoding for factor XII was a kind gift of F. Citarella (University of Rome “La Sapienza,” Italy). S. A. Newman (New York Medical College, Valhalla, USA) kindly provided the cDNA encoding for an N-terminal fragment of human fibronectin, comprising fibronectin type I domains 4-5.

Alternatively, recombinant finger domains of fibronectin (F4-5) and tPA were expressed with a His-tag. Two fibronectin F4-5 constructs were cloned. One construct comprising the Ig_{K} signal sequence (vector 71, ABC-expression facility, Utrecht University/UMC Utrecht). With two designed primers (8, 9, see FIG. 1A) the fibronectin fragment was obtained from the construct pcDNA3.1-Fn F4-5-GST and BamHI and NotI restriction sites were introduced at the termini. In addition, cDNA encoding for a C-terminal His-tag was included in the designed primer. The cDNA fragment was cloned BglII-NotI in vector 71 that was digested with BamHI-NotI. Vector 71 has a BamHI site next to the Ig_{K} signal sequence. See FIG. 1A for the construct details. A construct comprising the signal sequence of human growth hormone, the cDNA encoding for growth hormone (GH), an octa-His tag, a TEV cleavage site, the tPA F insert and a C-terminal hexa-His tag was made using vector 122b (ABC-expression facility). The tPA F-His cDNA was obtained using pcDNA3.1-tPA-F-GST as a template for a PCR with primers 10 and 11 (FIG. 1A). The PCR insert was digested BglII-NotI, the vector was digested BamHI-NotI. The BamHI site is located next to the GH-His-TEV sequence. A second Fn F4-5 construct was made similarly to the GH-His-tPA F-His construct.

The following part can roughly be divided into two parts. The first part describes criteria and means and methods in respect of the present invention and describes, for example, how to select and/or identify a pathogen which displays a cross-β structure comprising protein on its exterior, how to determine or verify whether a cross-β structure comprising protein is displayed. This part furthermore describes how to determine whether a cross-β structure is involved in pathogenicity and how to test whether, for example, the application of a cross-β structure binding compound results in reduced pathogenicity. In the second part some pathogens are subjected to the methods and means as described in the first part.

Role of cross-beta structure at the surface of pathogens in pathogenicity; leads for cross-beta structure binding compound-based therapies against infections

Pathogenicity of pathogens, such as bacteria, fungi, parasites and viruses, comes in several ways. The body reacts to pathogens with inflammatory responses and immunological responses. During infection components of the haemostatic system are also activated. It is disclosed herein that proteins with a cross-beta structure conformation at the surface of various pathogens mediate infection, including activation of components of the haemostatic system (Gebbink et al., 2005). A number of cell-based bioassays, blood enzyme activation tests and coagulation tests with a series of pathogens is conducted to provide evidence that compounds that interact with cross-beta structure are suitable to inhibit and/or prevent and/or counteract and/or abolish and/or reverse and/or diminish and/or interfere with infection and/or complications accompanied with or induced during infections. These examples have already provided (see below) and will further provide insight in the structure-function relationship of cross-beta structure during infection biology. The role of cross-beta structure is further assessed by including cross-beta structure binding compounds in the assays that may interfere with the pathogenic activity of the pathogens. Cross-beta structure binding compounds are used as potential inhibitory molecules in the in vitro (bio)assays and animal models. From these series of experiments it is concluded which cross-beta structure binding compounds act on cross-beta structure-mediated pathogenicity. The experiments provide leads for therapeutics for treatment of infections, based on cross-beta structure binding compounds.

A. Pathogens

The selection of pathogens that are analyzed for their pathogenic activity towards cells in our bioassays and for their influence on blood coagulation can be based on several criteria.

Some pathogens are known for their ability to bind and/or activate multiligand cross-beta structure binding proteins tissue-type plasminogen activator (tPA), factor XII and fibronectin (see Gebbink et al., 2005) and Tables 1 and 2 for pathogens that activate tPA or factor XII-mediated proteolytic processes). A series of observations in literature point to a role during infection for binding of cross-beta structure binding protein fibronectin to pathogens. For example, Spirochete Borrelia binding to subendothelial matrix was inhibited 48 to 63% by pretreatment of the matrix with anti-fibronectin antiserum. In addition, a 47 kDa fibronectin-binding protein expressed by Borrelia burgdorferi isolate B31 has been identified, and the cellular form of human fibronectin has been indicated as an adhesion target for the S fimbriae of meningitis-associated Escherichia coli. Moreover, the pavA gene of Streptococcus pneumoniae encodes a fibronectin-binding protein that is essential for virulence, and in Streptococcus pyogenes the gene of fibronectin-binding domain embp has been determined. Also for S. pyogenes several surface proteins are implicated in fibronectin binding, including protein F1, M and M-like. Curli fibers of Escherichia coli mediate internalization of bacteria by eukaryotic cells, and curli fibers bind fibronectin with high affinity. These binding and/or activation characteristics, together with binding studies performed with cross-beta structure binding dyes (Congo red, Thioflavin T) are considered to be a measure for the presence of proteins comprising cross-beta structure at the surface of the pathogens. As described before in this application, amyloid core proteins have been identified as being part of the core of several classes of pathogens. Those pathogens with cross-beta structure at their surface provide suitable models to analyze the role of cross-beta structure comprising proteins in the pathogenicity of these pathogens.

Alternatively, pathogen selection for our assays is driven by literature data showing pathogenicity of those pathogens towards cell types that are included in the assays, like, for example, cultured human umbilical vein endothelial cells (HUVEC), mouse microvascular bEnd.3 endothelial cells, THP-1 derived macrophages and/or monocytes, mouse bone marrow derived dendritic cells, human blood platelets and dendritic cells derived from human peripheral blood mononuclear cells. For example, for Staphylococcus aureus and Streptococcus pneumonia, both interaction with tPA and induction of platelet activation has been reported. Therefore, these pathogens provide suitable test cells for our analyses. For Staphylococcus aureus, binding of the fourth and fifth fibronectin type I domains of human fibronectin (Fn F4-5) has been demonstrated. Fn F4-5 are the domains that interact with a fibrin network and that bind to cross-beta structure in general.

Finally, selection criteria for pathogens that can be included in our further studies to unravel the role of pathogen surface proteins with cross-beta structure in host invasion, infection, immunity, inflammation and hemostasis is based on literature data describing the interaction of multiligand cross-beta structure receptors of a host with pathogens. For example, CD36 is an essential receptor on macrophages that mediates internalization of pathogens, for example, Staphylococcus aureus, and triggers signaling pathways resulting in tumor necrosis factor-α (TNF-α) and interleukin-12 (IL-12) expression. This makes Staphylococcus aureus an attractive model pathogen to study whether S. aureus core proteins with cross-beta structure are involved in the interaction with, e.g., CD36. For a first series of experiments, for example, a Streptococcus pneumonia strain is selected. Furthermore, for example, a Gram-positive bacterium is included in the studies, for example, a Staphylococcus strain is used, for example, a Staphylococcus aureus strain, for example, a Staphylococcus aureus Newman strain. In addition, a Gram-negative bacterium is included in the studies, for example, an Escherichia coli strain is used, for example, Escherichia coli strain TOP10 (Invitrogen, 44-0301) or, for example, Escherichia coli strain MC4100.

It is, therefore, clear for a skilled person that a suitable pathogen (i.e., a pathogen comprising cross-β structure on its exterior) may be selected in a variety of ways of which some have been non-limiting mentioned above.

B. Determination of the Presence of Cross-Beta Structure Protein Conformation on Pathogens

With the selected pathogens (see selection criteria above) a series of analyses is performed that provide insight in the presence of cross-beta structure protein conformation. Standard Congo red and Thioflavin T binding and fluorescence assays are conducted. For this, pathogens are incubated with the amyloid binding dyes and fluorescence enhancement is determined. Alternatively, pathogens are fixed, stained with the dyes and binding is analyzed under a fluorescence microscope or under a direct light microscope using polarized light (Congo red birefringence), or free pathogens are incubated with dyes and fixed afterwards, before microscopic analysis. Presence of cross-beta structure on pathogens may also be assessed by culturing pathogens on culture plates containing Congo red or Thioflavin T or Thioflavin S. Presence of cross-beta structure can be determined by visual inspection. Pathogen cells can also be analyzed by using electron microscopy, to determine the presence of fibrillar structures at the pathogen surface. Interaction with cross-beta structure binding proteins tissue-type plasminogen activator and factor XII is assessed using chromogenic assays. For this purpose, concentration series of the pathogens are mixed with 100-1000 pM tPA, 5-200 μg/ml plasminogen and 0.1-1 mM chromogenic plasmin substrate S2251 (Chromogenix), and conversion of plasminogen to plasmin upon tPA activation by cross-beta structure is followed in time during 37° C.-incubation. For factor XII activity measurement, concentration series of pathogen are mixed with 0.1-50 μg/ml factor XII, 0-5 μg/ml prekallikrein, 0-5 μg/ml high molecular weight kininogen and either chromogenic factor XII substrate S2222 (Chromogenix) for direct measurement of factor XII activity, or chromogenic kallikrein substrate Chromozym-PK (Boehringer-Mannheim) for indirect factor XII activity, and substrate conversion is followed in time spectrophotometrically during 37° C. incubation. Alternative to the tPA and factor XII activation assays, presence of cross-beta structure on pathogens may also be assessed using ELISA set-ups. For this purpose, for example, pathogens are immobilized onto the wells of ELISA plates. Subsequently, plates are blocked with a blocking solution, and concentration series of cross-beta structure binding compounds are added to the wells. Binding of the cross-beta structure binding compounds is determined using specific antibodies. Examples of cross-beta structure binding compounds that may be used for this approach are tPA, factor XII, fibronectin, finger domains derived from tPA, factor XII, fibronectin or hepatocyte growth factor activator (HGFA), soluble fragment of receptor for advanced glycation end products (sRAGE), soluble extracellular fragments of low density lipoprotein receptor related protein (LRP cluster 2, LRP cluster 4), (hybridoma) antibodies, intravenous immunoglobulins (IgIV or IVIg, either or not a fraction that is enriched by applying a cross-beta structure affinity column), or chaperones like, for example, BiP, HSP70, HSP90. All of the above-listed analyses are preferably performed with solutions before and after centrifugation for one hour at 100,000*g, or preferably before and after filtration using a 0.2 μm filter. Positive controls that are preferably included in the assays are glycated hemoglobin, amyloid-β and amyloid γ-globulins, prepared by incubation of β-globulins in H₂O at 37° C., after dissolving lypohilized γ-globulins in 1,1,1,3,3,3-hexafluoro-2-propanol and trifluoro acetic acid, followed by air-drying.

C. Cell Assays and Other Bioassays

1. General Overview of the Use of Bioassays

In all of the cell-based bioassays described below, cell concentration series of the selected pathogens (see above) are added to cultured cells to determine the optimal concentration for subsequent inhibitory studies. When suitable pathogen cell densities are determined, that induce a pathogenic response in the cultured cells, concentration series of putative blockers of the obtained pathogenic effects are tested by premixing concentrations of pathogen cells with concentration series of cross-beta structure binding compounds, and then by adding the premixes to cultured cells, followed by standard read-out measurements for pathogenicity. Alternatively, pathogens are first pelleted, solution with residual cross-beta structure binding compound is discarded, the pathogen cell pellet is resuspended in buffer and washed before applying the pathogen to the bioassay. In this way, information is obtained about the role of pathogen proteins comprising cross-beta structure in inducing pathogenic conditions in the cell cultures. Positive controls that are preferably included in the bioassays are glycated hemoglobin, amyloid-β, CpG, apoptotic cells of any kind, necrotic cells of any kind and lipopolysaccharide.

2. In Vitro Murine Dendritic Cell Assay

Immunity against pathogens is dependent on the presentation of antigens by antigen-presenting cells (APC), such as dendritic cells. Cultured murine dendritic cells (DCs) are applied as a model for immunogenicity of cross-beta structure bearing pathogens. For this purpose, DCs are isolated from the hind legs of, for example, 8 to 12-week-old Black-6 mice. Bones are isolated and rinsed in 70% ethanol, rinsed in RPMI-1640 medium with 25 mM HEPES, with 10% fetal calf serum, penicillin and Streptomycin. Then the bones are flushed with this buffer, in both directions. Eluates are cleared from erythrocytes by adding erythrocyte-specific lysis buffer (for example, obtained from the local UMC Utrecht Pharmacy Dept., catalogue number 97932329). Eluates are analyzed for viable cells by culturing them in cell culture plates. At this stage, the medium is enriched with 10 ng/ml GM-CSF. DCs grow in suspension or on a layer of macrophage cells. Using a FACS and specific antibodies, it is determined whether DCs are present and the activation state is analyzed. Preferably, the levels of cell surface receptors involved in endocytosis and co-stimulatory molecules, such as B7.1, B7.2, MHC class II, CD40, CD80, CD86 is determined on preferably CD11c-positive cells. Alternatively, activation of NF-κB and/or expression of cytokines will be used as indicators of activation of cells involved in immunogenicity, such as APC and DC. Preferably, the following cytokines are quantified: TNFα, IL-1, IL-2, IL-6, and/or IFNγ. Preferably, the cytokine levels are quantified by ELISA. Alternatively, the mRNA levels are quantified. For a person skilled in the art it is evident that function of APC and DC are tested as well.

Alternatively, a stable DC line or other antigen-presenting cells is used to test beneficial effects of depletion or neutralization of misfolded proteins with cross-beta structure on pathogens (Citterio et al., 1999).

3. In Vitro Generation of Peripheral Blood Human Monocyte-Derived Dendritic Cells and Activation Assay

Human DCs are generated from non-proliferating precursors selected from peripheral blood mononuclear cells (PBMCs), essentially by published methods (Sallusto and Lanzavechhia, 1994). In brief, the hematocrit fraction of freshly drawn citrated human blood or of buffy coat blood is used. Using the Ficoll/Lymphoprep-based separation-centrifugation method, PBMCs are separated. Subsequently, monocytes are purified from this PBMC fraction by using the Percoll-based separation-centrifugation and adherence method. The CD14-positive monocytes (0.5×10⁶/ml) are cultured for approximately six to seven days (37° C., 5% CO₂) in serum-free medium enriched with, for example, 10 ng/ml GM-CSF and 10 ng/ml IL-4. Presence of immature DCs is, for example, determined by Fluorescence Activated Cell Sorting (FACS) analysis for the presence or absence of CD14, CD1a, CD80, CD40, CD86, HLA-DR, CD83, CD206, CD36 and CD163 surface expression. Relative abundant presence of CD1a, CD36, CD40, CD86 and CD206 and relative low content of CD14-positive, CD80-positive, CD83-positive and CD163-positive cells will serve as a quality measure for the immature DCs. After obtaining the immature DCs upon stimulation with GM-CSF and IL-4, cells are, for example, be incubated for 16 to 72 hours with a concentration series of cultured Streptococcus pneumoniae, Escherichia coli or Staphylococcus aureus Newman in PBS or in buffer comprising cross-beta structure binding compounds like, for example, Congo red, Thioflavin T, tPA, finger domains and IgIV. To determine the influence of the bacterial cells on the DCs, typically surface density of CD83, CD86, CD80, CD163, CD14, CD40, CD36, scavenger receptor A, LRP, CD1a, HLA-DR, LOX-1, Toll-like receptor-2 (TLR2), TLR4, TLR9 and mannose receptor/CD206 and/or the percentage of positive cells with respect to the DC surface molecules are, for example, measured using FACS.

4. In Vitro Human Umbilical Vein Endothelial Cell and Murine Microvascular bEnd.3 Endothelial Cell Activation Assay

Glycated proteins comprising a cross-beta structure and amyloid-β induce inflammatory responses and are believed to contribute to the pathogenesis of certain protein misfolding diseases (diabetes type II, Alzheimer's disease). In general, misfolded proteins induce cellular dysfunction with enhanced expression or activation of inflammatory signals. The effect of misfolded proteins on endothelial cell (dys)function is, for example, measured by determining the levels of reactive oxygen species or nitric oxide or tissue factor in response to misfolded proteins. Human umbilical vein endothelial cells (HUVEC) that are isolated and cultured, according to standard protocols, are used, or other endothelial cells such as the murine microvascular bEnd.3 endothelial cell line. The levels of reactive oxygen species (ROS), like, for example, nitric oxide, are monitored using fluorescent probes, such as CM-H2DCF-DA. Alternatively, cell viability is monitored by standard MTT-assay. The levels of tissue factor expression is determined using a chromogenic assay with purified factor VII, purified factor X and chromogenic substrate S-2765 (Chromogenix), using cell lysates. For example, bEnd.3 cells are seeded at 120,000 cells/well of a 96-well culture dish, and cultured overnight. Cells are subsequently exposed to a dilution series of overnight cultured E. coli cells or S. aureus cells, which are resuspended in PBS after centrifugation and discarding the Luria broth supernatant, or which are resuspended in a solution comprising cross-beta structure binding compounds Thioflavin T (Sigma-Aldrich), Congo red (Sigma-Aldrich), tissue-type plasminogen activator (Actilyse, Boehringer-Ingelheim) and intravenous immunoglobulins (IgIV, Octagam, Octapharma). Changes in levels of ROS are followed in time during a one-hour incubation at 37° C., at, for example, two-minute intervals.

The cultured primary cells and the cell line provide the opportunity to perform in vitro cell assays that are accepted in research community as model systems for certain disease states.

5. Phagocytosis of Cross-Beta Structure Comprising Pathogens

The uptake of cross-beta structure comprising pathogens, and the effect of cross-beta structure binding compounds are studied in vitro using cultured cells, preferably monocytes, dendritic cells, or macrophages or similar cells, for example, U937 or THP-1 cells. Preferably, cross-beta structure comprising pathogens are labeled, preferably with 125I or a fluorescent label, preferably FITC, covalently attached to the molecule by a linker molecule, preferably ULS (universal Linkage system) or by applying an alternative coupling method. Cells are preferably labeled with mepacrin or other fluorescent labels, such as rhodamine. Phagocytic cells are incubated in the presence of labeled cross-beta structure comprising cells in the presence or absence of a cross-beta structure binding compound (see below). After incubation, preferably during several hours, the uptake of labeled molecules or cells is measured preferably using a scintillation counter (for 125I) or by FACS-analysis (with fluorescent probes) or immunofluorescent microscopy. The uptake of pathogen cells is also counted under a light microscope with visual staining of these cells.

Alternatively, the response of cells that are involved in phagocytosis to cross-beta structure comprising pathogens is also assessed by measuring expression levels of several markers for an inflammatory/activation/thrombogenic response. Using commercially available ELISAs, expression levels of tumor necrosis factor-α and interleukin-8 are determined upon exposure of, for example, macrophages to cross-beta structure comprising pathogens. Expression levels of tissue factor are determined using a chromogenic assay with chromogenic substrate S2765 (Chromogenix), factor VII and factor X.

6. Ex vivo Human Blood Platelet Aggregation Assay

The influence of cross-beta structure binding compounds on blood platelet aggregation induced by cross-beta structure comprising pathogens is tested with washed platelets in an aggregometric assay. Freshly drawn human aspirin free blood is mixed gently with citrate buffer to avoid coagulation. Blood is spinned for 15 minutes at 150*g at 20° C. and supernatant is collected; platelet rich plasma (PRP). Buffer with 2.5% trisodium citrate, 1.5% citric acid and 2% glucose, pH 6.5 is added to a final volume ratio of 1:10 (buffer-PRP). After spinning down the platelets upon centrifugation for 15 minutes at 330*g at 20° C., the pellet is resuspended in HEPES-Tyrode buffer pH 6.5. Prostacyclin is added to a final concentration of 10 ng/ml, and the solution is centrifuged for 15 minutes at 330*g at 20° C., with a soft brake. The pellet is resuspended in HEPES-Tyrode buffer pH 7.2 in a way that the final platelet number is adjusted to 200,000 to 250,000 platelets/μl. Platelet counts are adjusted to approximately 300,000 platelet/μl when PRP is used. Platelets are kept at 37° C. for at least 30 minutes, before use in the assays, to ensure that they are in the resting state. Platelets of approximately five donors are isolated separately.

For the aggregometric assays, platelet solution is added to a glass tube and prewarmed to 37° C. A stirring magnet is added and rotation is set to 900 rpm, and the apparatus (Whole-blood aggregometer, Chrono-log, Havertown, Pa., USA) is blanked. A final volume of 1/10 of the volume of the platelet suspension is added (typically 300 μl to 300 μl platelet suspension), containing the agonist of interest and/or the premixed antagonist of interest, prediluted in HEPES-Tyrode buffer pH 7.2. Alternatively, pathogens are first pelleted, solution with residual cross-beta structure binding compound is discarded, the pathogen cell pellet resuspended in buffer and washed before applying the pathogen to the bioassay. Aggregation is followed in time by measuring the absorbance of the solution that will decrease in time upon platelet aggregation. As a positive control, either 10 μg/ml collagen (Kollagenreagens Horm, NYCOMED Pharma GmbH, Linz, Austria; lot 502940), or 5 μM of synthetic thrombin receptor activating compound TRAP, or 10-100 μg/ml glycated hemoglobin or 10-100 μg/ml amyloid-β is used. Aggregation is recorded for at least 15 minutes.

7. Ex Vivo Human Plasma Coagulation Assays

For analysis of the influence of cross-beta structure comprising pathogens on the characteristics of blood coagulation, and for analysis of the effects of cross-beta structure binding compounds on the influence of pathogens on coagulation, two standard coagulation tests are performed on, for example, a KC10 Coagulometer. Pooled human plasma of approximately 40 apparently healthy donors is clotted by adding either negatively charged phospholipids, CaCl2 and kaolin when an activated partial thromboplastin time (aPTT) is considered, or tissue factor rich thromboplastin and CaCl₂ when prothrombin time (PT) determinations are considered. APTTs and PTs are performed as follows. Plasma is incubated with concentration series of pathogen for, for example, 15 minutes to 120 minutes at room temperature or at 37° C. Pathogen cells are pelleted by centrifugation, for example, for 30 seconds with 16,000*g, and plasma supernatant is subsequently applied in either an APTT or a PT. At conditions that influence the coagulation tests, preincubations of pathogens with concentration series of cross-beta structure binding compounds are performed, before applying the pathogens to plasma, or in an alternative way, pathogens and cross-beta structure binding compounds are applied to plasma together. For an APTT analysis, 50 μl of plasma is mixed with 50 μl of a physiological buffer. Next 25 μl of 900 μg/ml Kaolinum Ponderosum (Genfarma) and 120 μM lipid vesicles (phosphatidyl serine/phosphatidyl choline/phosphatidyl ethanolamine) in a 20/40/40% (v/v) ratio are added, and the mixture is prewarmed to 37° C. To start the assay, 25 μl of a 50 mM CaCl₂ solution is added. For a PT analysis, 50 μl of (pretreated) plasma is combined with 50 μl H₂O and is incubated for five minutes at 37° C. The PT analysis is started by adding 50 μl of a Thromborel S stock, which is prepared at twice the concentration as recommended by the manufacturer (DADE Behring).

8. In Vitro Murine Monocyte Tissue Factor, Tumor Necrosis Factor-α and Interleukin-8 Expression

THP-1 cells are cultured using conditions that provide monocytes. For this purpose we culture the cells in Iscove's Modified Dulbecco's Medium (IMDM) with 5% fetal calf serum and 50 μg/ml gentamycin. For further studies THP-1 cells are also stimulated and differentiated by exposing the cells to, for example, phorbol 12-myristate 13-acetate (PMA) and/or Tetra-Phorbol-Acetate (TPA) and/or interferon-γ and/or lipopolysaccharides.

Analysis of Tissue Factor Expression by THP-1

For tissue factor expression analysis purposes, THP-1 cells are cultured in IMDM without gentamycin and streptomycin. At day 0, one ml of cells is seeded at 1×10⁶ cells/ml in the wells of six-well culture plates. At day 1, cells are stimulated with putative agonists and/or antagonists for six hours at 37° C. (regular culturing conditions). Positive control is a concentration series of LPS, negative control is buffer. Agonists that are tested are misfolded proteins comprising cross-beta structure and pathogens comprising amyloid core proteins with cross-beta structure and apoptotic cells and necrotic cells. Antagonists that are tested are inhibitory antibodies against THP-1 surface receptors involved in signal transduction upon exposure to cross-beta structure, like, for example, antibodies against CD91/LRP, CD36, receptor for advanced glycation end products (RAGE), scavenger receptor A, scavenger receptor B-I, Toll-like receptor 4. Other antagonists that are included for analysis of inhibitory properties are cross-beta structure binding compounds like, for example, Congo red, Thioflavin T, tPA, fibronectin, BiP, HSP60, HSP70, HSP90, gp96, soluble fragments of LRP (cluster 2, cluster 4), soluble fragment of RAGE, finger domains, antibodies, IgIV. After stimulation, cells are pelleted by five minutes centrifugation at low speed. Supernatants are analyzed for TNF-α and/or IL-8 levels, using commercially available ELISAs. The cell pellet is resuspended in 100 μl TBS (50 mM Tris-HCl, 150 mM NaCl, pH 7.0-7.3). Next, the cells are frozen and thawed for four subsequent cycles. Cells are centrifuged for ten minutes at 16,000*g and the supernatant is used for further analysis. First, protein concentrations are determined using a regular protein concentration assay like, for example, a Bicinchoninic Acid (BCA) Protein Assay. Protein concentrations are equalized between samples with TBS to correct for variations in cell density. For analysis of tissue factor levels, 50 μl of cell lysate is incubated with 50 μl buffer comprising 10 μg/ml factor X, 5 U/ml factor VII (FVII) and 5 mM CaCl₂, for 45 minutes at 37° C. in wells of a 96-well plate. Then, 50 μl of a 4.5 mM chromogenic activated factor X substrate S2765 (Chromogenix) stock is added. Conversion of the substrate by activated factor X at 37° C. is recorded in time for at least ten minutes, by absorbance readings at 405 mm.

D. Cross-Beta Structure Binding Compounds Used as Potential Inhibitors of Cross-Beta Structure-Mediated Pathogenicity of Pathogens

To be able to analyze the role of cross-beta structure comprising proteins on pathogens in pathogenicity, as assessed in the above-listed series of bioassays, a series of cross-beta structure binding compounds is included in the assays as potential inhibitors of cross-beta structure-mediated pathogenicity. Cross-beta structure binding compounds are typically included in the assays at concentrations of 1-5000 μg/ml, or 1 nM —1 mM. Examples of cross-beta structure binding compounds that are used are Congo red, Thioflavin T, Thioflavin S, tPA, factor XII, fibronectin, finger domains derived from tPA, factor XII, fibronectin or HGFA, sRAGE, sLRP, LRP cluster 2, LRP cluster 4, (hybridoma) antibodies, IgIV (either or not a fraction that is enriched by applying a cross-beta structure affinity column), soluble extracellular fragment of LOX-1, soluble extracellular fragment of CD40, soluble extracellular fragment of CD36, or molecular chaperones like, for example, clusterin, haptoglobin, BiP/grp78, HSP60, HSP70, HSP90, gp96 (see Tables 3 through 5 for more examples of cross-beta structure binding compounds).

### Materials & Methods

A. Culturing of Staphylococcus aureus Newman and Escherichia coli TOP10, and Preparing Reference and Tester Cell Samples

Staphylococcus aureus Newman, which was a kind gift of Dr. Jos van Strijp and Dr Kok van Kessel (Dept. of Microbiology, University Medical Center Utrecht, the Netherlands) was plated on a blood plate from a stock stored at −70° C., and incubated overnight at 37° C. The plate was stored at 4° C. Escherichia coli strain TOP10 (Invitrogen, 44-0301) was plated on agar with Luria broth medium from a −80° C. glycerol stock, and incubated overnight at 37° C. The plate was stored at 4° C. Overnight cultures of 5 ml in Luria broth medium were grown at 37° C. with vigorous shaking and aeration, by streaking a single colony with the tip of a pipet and transferring the tip to the medium in a 15-ml tube. The cell density in overnight cultures was determined by measuring the absorbance at 600 nm (A₆₀₀=1 is equivalent with 0.8×10⁹ cells/ml). Cells were pelleted by centrifugation for one minute at 16,000*g or for ten minutes at 3,000*g. Medium was discarded. One half of the cells were resuspended in PBS in 1/10 of the original medium volume (10× concentration of the cells) by pipetting and swirling. These cells were used as reference cells. The second half of the cells was designated as “tester” cells and was resuspended in PBS with 5 mM Thioflavin T (again 1/10 of the original medium volume), by pipetting and swirling, as with all subsequent handlings. The tester cells were incubated for five minutes at room temperature with constant swirling. Cells were pelleted by centrifugation for 30 seconds at 16,000*g and supernatant was discarded. Cells were resuspended in 5 mM Congo red in PBS and incubated in a way similar to the Thioflavin T incubation. After pelleting the cells, they were resuspended in PBS and again pelleted by centrifugation for 30 seconds at 16,000*g. Supernatant was discarded. Finally, tester cells were resuspended in a solution of 25 μM tissue-type plasminogen activator (Actilyse, Boehringer-Ingelheim) and 25 mg/ml intravenous immunoglobulins (IgIV, Octagam, Octapharma), in approximately 1/150 of the original medium volume (75× concentration of the cells). After a 30-minute incubation, cells were pelleted, resuspended in PBS (approximately 1/10 of the original medium volume) and kept at room temperature for use at the same day or kept at 4° C. for later use within 72 hours E. coli cell suspension was yellow-light orange after all subsequent incubations, S. aureus cell suspension was red. Initial cell densities of the overnight cultures were 1.8×10⁹ cells/ml for the S. aureus and 1.3×10⁹ for the E. coli cells. The work suspensions had cell densities of 1.8×10¹⁰ cells/ml and 1.8×10¹⁰ cells/ml, respectively.

Reference and tester Staphylococcus aureus Newman cells or Escherichia coli TOP10 cells obtained as described above were applied in a series of bioassays. Cells at various indicated densities were analyzed for their activity in the assays C-3 (activation of human DCs), C-4b (ROS production in murine ECs), C-6 (platelet aggregation), C-7 (plasma coagulation, PT and aPTT analyses) and C-8 (tissue factor expression in THP-1 monocytes), as described above. In addition, plasmin generation in a chromogenic tPA/plasminogen activation assay will be assessed and activation of factor XII and prekallikrein will be assessed in a chromogenic assay. For this purpose dilution series of the pathogen cells will either be mixed with final concentrations of 400 pM tPA, 0.2 μM plasminogen (purified from human plasma) and 0.8 mM chromogenic plasmin substrate S2251 (Chromogenix) or chromogenic plasmin substrate Biopep-1751 (Biopep, France) in a physiological buffer, or with 0.3 mM chromogenic kallikrein substrate Chromozym-PK (Roche Diagnostics, Almere, The Netherlands), 1 μg/ml zymogen factor XII (#233490, Calbiochem, EMD Biosciences, Inc., San Diego, Calif.), human plasma prekallikrein (#529583, Calbiochem) and human plasma cofactor high-molecular weight kininogen (#422686, Calbiochem). For the factor XII assay, the assay buffer contained HBS (10 mM HEPES, 4 mM KCl, 137 mM NaCl, pH 7.2). Plasmin or kallikrein generation will be followed in time upon 37° C. incubation, by measuring the A₄₀₅ absorbance each minute for two to three hours. Buffer will serve as a negative control, concentration series of glycated hemoglobin and/or of amyloid γ-globulins, prepared as described above, and/or 150 μg/ml kaolin will serve as positive controls.

C4. Production of Reactive Oxygen Species by Mouse Microvascular bEnd.3 Endothelial Cells

To assess production of reactive oxygen species by cultured mouse microvascular bEnd.3 endothelial cells (ECs), cells are seeded at 128,000 cells/well of a 96-well plate (Costar, 3904). After adherence for six hours, cells are washed twice with PBS and cultured overnight in DMEM with 0.1% bovine serum albumin (DMEM from Gibco with 4500 mg/l glucose, GlutaMAX and pyruvate, enriched with 100 μg/ml penicillin and streptomycin and 10% fetal calf serum). Cells are subsequently washed once with PBS enriched with 1 mM CaCl₂, 0.5 mM MgCl₂ and 0.1% w/v glucose (“enriched PBS”) and incubated for 30 minutes at 37° C. in the dark with 75 μl of CM-H₂DCFDA (Invitrogen C6827) from a 10 μM stock in PBS. Then, cells are washed twice with enriched PBS and incubated for 15 minutes at 37° C. in the dark, either with 190 μl enriched PBS, or 190 μl enriched PBS with 1 μM Nω-Nitro-L-arginine methyl ester hydrochloride. For the analysis of ROS production, 10 μl of tester samples and controls are added to separate wells, and fluorescence is measured every two minutes for 70 minutes upon excitation at 488 nm with the emission wavelength set to 538 nm.

In one series of experiments, bEnd.3 cells were exposed to 160× diluted stocks of E. coli TOP10 or S. aureus (final cell densities 8.1×10⁷ cells/ml and 1.13×10⁸ cells/ml, respectively) in buffer or in buffer with either 1.25 mg/ml IgIV (Octagam), or finger domains (see below), or 220 μM Congo red, or 220 μM Thioflavin T (ThT), or 1.1 μM tPA, and ROS levels were followed in time upon 37° C. incubation. The bacteria were pre-incubated with PBS or the cross-beta structure binding compounds at concentrations of 25 mg/ml IgIV, 0.8 mg/ml finger domains, 4.4 mM Congo red, 4.4 mM Thioflavin T, or 22 μM tPA, respectively, for approximately one hour at room temperature. Subsequently, the bacterial cell suspensions were diluted twenty-fold in the cell culture medium with the ECs. As a source of finger domains, a mixture was prepared consisting of recombinant human tPA finger (F) with a C-terminal His-tag which was expressed in Saccharomyces cerevisiae (Biotechnology Application Center (BAC-Vlaardingen/Naarden, The Netherlands), a chemically synthesized hepatocyte growth factor activator (HGFA) finger domain (Dr. T. Hackeng, Academic Hospital Maastricht, the Netherlands) and recombinant human fibronectin finger domain tandem 4 and 5 with a C-terminal His-tag which was expressed in HEK 293E cells (ABC-expression facility, Utrecht). The cDNA constructs were prepared following standard procedures known to a person skilled in the art. Domain boundaries of fibronectin F4-5 and tPA F were taken from the human fibronectin and human tPA entries in the Swiss-Prot database (P02751 for fibronectin, P00750 for tPA) and comprised amino-acids NH₂—I182-V276—COOH of fibronectin and NH₂—G33-S85—COOH of tPA. Affinity purification of the expressed proteins was performed using His₆-tag—Ni2+ interaction and a desalting step. For HGFA, residues 200 to 240 (Swiss-Prot entry Q04756) were taken. Stock solutions of fibronectin F4-5, tPA F and HGFA F were mixed to final concentrations of 0.9 mg/ml, 0.7 mg/ml and 1.25 mg/ml, respectively. The final concentration of finger domains is approximately 0.8 mg/ml.

C6. Induction of Platelet Aggregation by Pathogens

The influence of S. aureus Newman bacterium cells and E. coli TOP10 bacterium cells on blood platelet aggregation was tested with washed platelets (platelet rich plasma, PRP) in an aggregometric assay. Freshly drawn human aspirin free blood was mixed gently with citrate buffer to avoid coagulation. Blood was spinned for 15 minutes at 150*g at 20° C. and supernatant was collected; platelet rich plasma (PRP) with an adjusted final platelet number of 300,000 platelets/μl. Platelets were kept at 37° C. for at least 30 minutes, before use in the assays, to ensure that they were in the resting state. Platelets of two donors were isolated separately on different days.

For the aggregometric assays, 270 μl platelet solution was added to a glass tube and prewarmed to 37° C. A stirring magnet was added and rotation was set to 900 rpm, and the apparatus (Whole-blood aggregometer, Chrono-log, Havertown, Pa., USA) was blanked. A final volume of 30 μl was added, containing the agonist of interest (pathogen) and/or the premixed antagonist of interest (pathogen pretreated with cross-beta structure binding molecules), prediluted in HEPES-Tyrode buffer pH 7.2. Final S. aureus concentration was 1.8×10⁹ cells/ml, for E. coli 1.3×10⁹ cells/ml. Aggregation was followed in time by measuring the absorbance of the solution that will decrease in time upon platelet aggregation. As a positive control, 5 μM of synthetic thrombin receptor activating peptide TRAP was used. Aggregation was recorded for 15 minutes and expressed as the percentage of the transmitted light (0-100%).

Analysis of Bacterium Cell Vitality After Exposure to Cross-Beta Structure Binding Compounds

S. aureus and E. coli cells were treated with PBS or cross-beta structure binding compounds according to the above-given description. After overnight storage of the cell preparations in PBS at 4° C., vitality of the cells was assessed by inoculating 1 ml of LB medium for six hours with vigorous shaking and aeration with 25μl cell suspension. After six hours, cell density was determined by an absorbance reading at 600 nm with 50× diluted cell cultures in PBS. Starting cultures contained 3.25×10⁸ E. coli cells/ml and 4.5×10⁸ S. aureus cells/ml.

B. Activation of the Contact System of Coagulation by E. coli with Amyloid Curli

E. coli strain MC4100 was grown using two different conditions on agar with colonization stimulating factor (CFA), using protocols known to a person skilled in the art. E. coli on one plate were grown for approximately 44 hours at 26° C. to induce expression of amyloid curli core protein comprising cross-beta structure. A second plate was cultured for 24 hours at 37° C. which suppresses curli expression. Cells were scraped from the plates and suspended in PBS. Cell density was measured and equalized. The two E. coli preparations were tested for their ability to activate factor XII and prekallikrein in an in vitro assay for determination of contact system of coagulation-activating properties. For this purpose an E. coli density of 2.08×10⁹ cells/ml was used in the assay, that was performed as described above.

C3. In Vitro Generation of Human Blood Derived Dendritic Cells and Activation Assay

To investigate the influence of pathogen cells comprising cross-beta structure core proteins on human DCs, the DCs were generated from non-proliferating precursors in peripheral blood mononuclear cells (PBMCs), essentially by established methods (Sallusto and Lanzavecchia, 1994). Briefly, PBMCs from buffy coat blood (Sanquin Blood Bank, Utrecht, The Netherlands) were purified using Lymphoprep (1.077 g/ml; Axis-Shield, Oslo, Norway) centrifugation. Monocytes were purified from PBMC by using Percoll (1.131 g/ml; Amersham Biosciences, Upsalla, Sweden) gradient centrifugation consisting of three layers (1.076, 1.059, and 1.045 g/ml). The low density monocyte-enriched fraction was collected, and subsequently seeded at 0.5×10⁶/ml in CellGro DC serum-free medium (CellGenix, ITK Diagnostics, Uithoorn, The Netherlands) in polystyrene 175 cm² culture flasks. After 45 minutes at 37° C. and 5% CO₂, non-adherent cells were discarded. Adherent cells (monocytes>90% CD14 positive) were cultured in CellGro DC medium containing 10 ng/nl GM-CSF (Tebu-Bio, Heerhugowaard, The Netherlands) and 10 ng/ml IL-4 (Tebu-Bio) for six days. Fresh GM-CSF and IL-4 were added every two days. After six days at 37° C. and 5% CO₂, the non-adherent cell fraction was harvested and counted. FACS analysis using the following markers was performed (FACS: anti-CD14, anti-CD1a, anti-CD80, anti-CD40, anti-CD86, anti-HLA-DR, anti-CD83, anti-CD206, anti-CD36 and anti-CD163 (all these fluorescein isothiocyanate (FITC)- and phycoerythrin (PE)- conjugated anti-CD markers were purchased from BD Biosciences, Erembodegem, Belgium), to establish whether immature DCs have been generated. Percentages of cells that were positive for the listed markers were 2% for CD14, 97% for CD1a, 4% for CD80, 69% for CD40, 69% for CD86, 60% for HLA-DR, 1% for CD83, 95% for CD206, 23% for CD36 and <1% for CD163, indicating that immature DCs have indeed been obtained. These immature DC were suspended to a final cell density of 1×10⁶ cells/ml in CellGro DC medium, and 1 ml was transferred to low-adherent polypropylene 5-ml tubes. Maturation assays were started by adding 10 μl of a tester solution (1:100 dilution). Dilution series of E. coli TOP10 and S. aureus Newman were added to DCs and pathogen densities ranged from 5.1×10⁴ to 3.25×10⁶ cells/ml for the E. coli, and from 7.0×10⁴ to 4.5×10⁶ cells/ml for the S. aureus (two-fold dilution series in seven stages). The E. coli and S. aureus were either pre-incubated with PBS, or with a serial series of cross-beta structure binding compounds comprising ThT, Congo red and tPA+IgIV, as described above. As the negative control DCs were incubated in plain medium. DCs were stimulated for 20 hours at 37° C., 5% CO₂. After this 20-hour incubation time, DCs were analyzed by FACS for the percentage of CD36, CD40 and CD206-positive cells, and for these markers the ratios between the geometric Mean Fluorescence Intensity (MFI) of a specific signal and the MFI of the accompanying noise was determined (MFI ratios). Cell morphology was assessed by analyzing forward scatter measurements and side or orthogonal scatter measurements (dead cell and contaminating lymphocyte fractions were excluded from the analysis).

Analysis of Tissue Factor Expression by THP-1 upon Stimulation with S. aureus that were Pre-Incubated with Buffer or Cross-Beta Structure Binding Compounds

For tissue factor expression analysis purposes, THP-1 cells were cultured in IMDM without gentamycin and streptomycin. At day 0, one ml of cells was seeded at 1×10⁶ cells/ml in the wells of six-well culture plates. At day 1, cells were stimulated for six hours at 37° C. with S. aureus that were pre-incubated with PBS or with cross-beta structure binding compounds ThT, Congo red, tPA and IgIV, as described above, at a cell density of 1.8×10⁷/ml (regular culturing conditions). Negative control was buffer. After six hours, the cells were pelleted by centrifugation and resuspended in 100 μl TBS (50 mM Tris-HCl, 150 mM NaCl, pH 7.0-7.3). Next, the cells were frozen and thawed for four subsequent cycles. Cells were centrifuged for ten minutes at 16,000*g and the supernatant was used for analysis of tissue factor (TF) expression. First, protein concentrations were determined using an established protein concentration assay (Bicinchoninic Acid (BCA) Protein Assay). Protein concentrations were equalized between samples with TBS to correct for variations in cell density. For analysis of TF levels, 50 μl of cell lysate was mixed with 50 μl TBS comprising 10 μg/ml factor X, 5 U/ml FVII, and 5 mM CaCl₂, and 50 μl of a 4.5 mM stock of chromogenic activated factor X substrate S2765 in H₂O, in wells of a 96-well plate. Conversion of the substrate by activated factor X at 37° C. was recorded in time for 100 minutes, by absorbance readings at 405 nm. As an additional control, factor X activity was assessed with S. aureus cells only, omitting the monocytes.

In a second series of experiments THP-1 monocytes were incubated in a similar manner with 40 μg/ml glycated hemoglobin or 10 μg/ml amyloid-β(1-40). Negative control was buffer, positive control was 10 μg/ml lipopolysaccharide.

Results

C4. In Vitro Murine bEnd.3 Endothelial Cell Activation Assay: ROS Production (I)

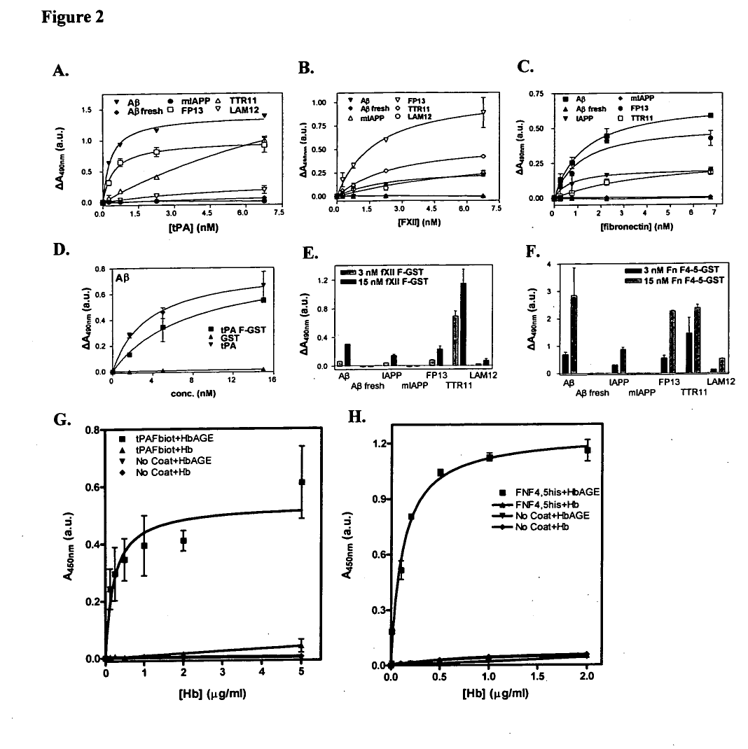

To determine whether cross-beta structure binding compounds Thioflavin T, Congo red, tissue-type plasminogen activator (tPA) and IgIV are able to reverse pathogenic effects of pathogens on ECs, bEnd.3 cells were exposed to 6×10⁸ E. coli TOP10 cells/ml and ROS production by the ECs was measured in time. For this purpose, bEnd.3 cells were cultured overnight at a density of 128,000 cells/well of a 96-well plate. The overnight grown E. coli cells were either resuspended in PBS before 20× dilution in cell culture medium at 1.2×10⁹ cells/ml (20× stock), or resuspended in 2.5 mM Congo red and 5 mM Thioflavin T in PBS after centrifugation and discarding the LB medium, incubated for ten minutes at room temperature with swirling, pelleted and dissolved at 1.2×10⁹ cells/ml (20× stock) in 25 μM tPA and 25 mg/ml IgIV by swirling. As an example, binding of IgIV to cross-beta structure is shown for glycated albumin (FIG. 3A). BEnd.3 cells were incubated with PBS or with 100 μM H₂O₂ as negative and positive controls for ROS induction, respectively (FIG. 3B). FIG. 3B shows the increased ROS production when bEnd.3 cells are exposed to E. coli cells. Pre-incubation of E. coli cells with Congo red, Thioflavin T, tPA and IgIV reduces ROS production significantly (FIG. 3B). This inhibition of ROS production by bEnd.3 ECs upon exposure to E. coli cells that are pre-incubated with cross-beta structure binding compounds show a role for the cross-beta structure at the surface of the E. coli cells in mediating pathogenic effects on ECs.

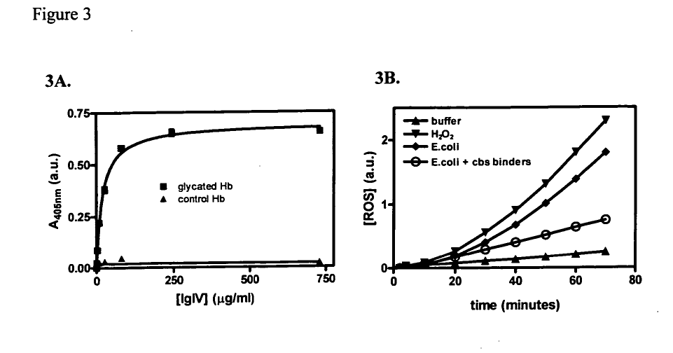

In a second series of experiments, E. coli TOP10 cells were pre-incubated in a serial set-up with PBS comprising first 2.5 mM Thioflavin T, then 5 mM Congo red and finally a mixture of 25 μM tPA and 25 mg/ml IgIV, respectively. Control cells were kept in PBS. Finally, E. coli cells were resuspended in PBS at 1.3×10¹⁰ cells/ml. Notably, pelleted cells appeared brownish-yellow and the cell suspension was orange-yellow after the incubations with cross-beta structure binding compounds, whereas the control cells in PBS were light-brown. To determine the influence of the two E. coli preparations on ROS production by bEnd.3 ECs, 8×10⁷ E. coli cells/ml were exposed to the ECs. In FIG. 4A, increased ROS production upon stimulation of ECs with the control E. coli cells is observed. Pre-treatment of the E. coli with cross-beta structure binding compounds decreases the potency to induce ROS production (FIG. 4A). Again, the ability of cross-beta structure binding compounds to reverse adverse effects of E. coli cells towards ECs show a role of bacterium cell surface proteins with cross-beta structure conformation in pathogenicity.

B. Factor XII/Prekallikrein Activation by E. coli

To test the potency of E. coli cells to induce factor XII/prekallikrein activation, cells at 1.3×10⁷ cells/ml were tested in a chromogenic factor XII activation assay using chromgenic kallikrein substrate Chromozym-PK. Activation of factor XII to factor XIIa and subsequently prekallikrein to kallikrein is observed upon incubation of the proteins with E. coli cells (FIG. 4B). Pre-treatment of the cells with PBS containing first 5 mM Thioflavin T, then 5 mM Congo red and finally a mixture of 25 μM tPA and 25 mg/ml IgIV resulted in background factor XII activation, similar to when buffer is used as a negative control. These results show that incubation of E. coli with cross-beta structure binding compounds reduces the potency to activate the intrinsic pathway of coagulation in hemostasis.

C6. Induction of Platelet Aggregation by Pathogens

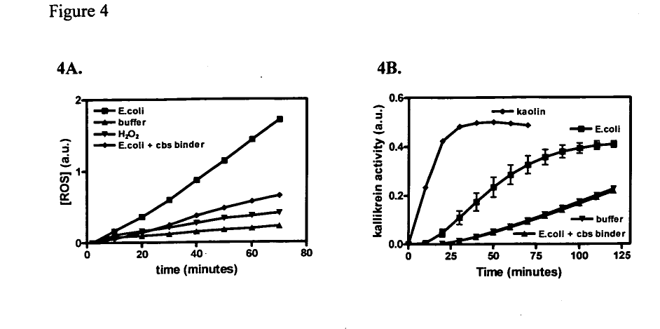

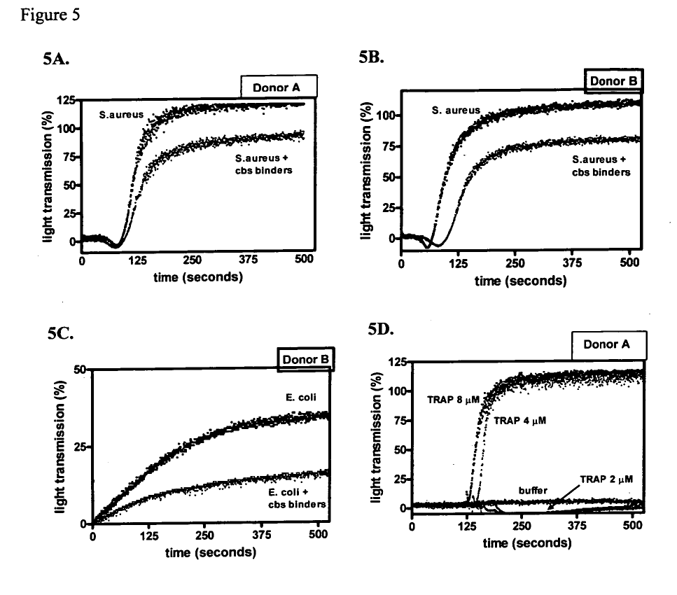

PRP was obtained from blood obtained from the local UMC Utrecht mini donor facility. Introduction of 1.8×10⁹ S. aureus Newman cells/ml or 1.3×10⁹ E. coli TOP10 cells/ml in PRP readily results in platelet aggregation (FIG. 5). The S. aureus is a more potent stimulator of platelet aggregation than the E. coli strain. Both PRP of donor A and B respond similarly to S. aureus, whereas only PRP obtained from donor A was activated by E. coli. Pre-treatment of the S. aureus cells and the E. coli cells with respectively 2.5 mM Thioflavin T, 5 mM Congo red, and 25 μM tPA+25 mg/ml IgIV inhibits pathogen induced cell aggregation (FIG. 5). These data disclose a role for surface exposed amyloid-like core proteins in induction of platelet aggregation. Apparently, binding of cross-beta structure binding compound prevents the activity of the pathogen-associated misfolded protein in part.

Vitality of Bacteria Before and After Treatment with Cross-Beta Structure Binding Compounds

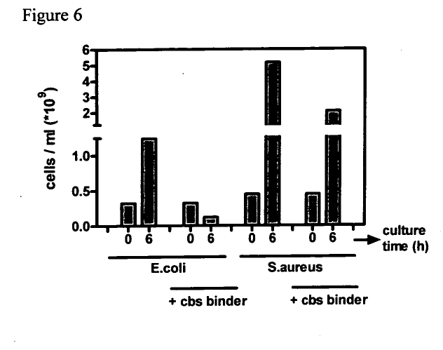

In FIG. 6, cell densities of E. coli TOP10 and S. aureus Newman cultures are given that were started with inoculum of cells that were either pretreated with PBS, or with cross-beta structure binding compounds Thioflavin T, Congo red, tPA and IgIV. Vitality of E. coli that was treated with cross-beta structure binding compounds is diminished to a large extent. The cell density after six hours of culturing is even lower than could be expected based on the cell density at the start. For the S. aureus cultures it is clear that cell vitality is also affected by the treatment with cross-beta structure binding compounds. The cell density in the culture started with S. aureus that was pretreated with cross-beta structure binding compounds is approximately 40% when compared to the cell density in the culture started with control cells. These results indicate that either exposure and/or binding of bacteria to cross-beta structure binding compounds hampers cell growth of vital cells, or that exposure and/or binding of bacteria to cross-beta structure binding compounds kills and/or inactivates the cells, thereby reducing the number of vital cells that could divide/growth. Therefore, these results show a beneficial role for cross-beta structure binding compounds in the treatment of infections by reducing pathogenicity of pathogens with amyloid-like cross-beta structure comprising core proteins.

B. Binding of Cross-Beta Structure Binding Compounds to E. coli TOP10 and S. aureus Newman

Binding of cross-beta structure binding compounds to E. coli TOP10 and S. aureus Newman after incubation of the bacteria with Thioflavin T, Congo red, tPA and IgIV, as described in the Materials & Methods section, was determined in two ways. First, binding of cross-beta structure binding dyes Thioflavin T (yellow) and Congo red (red) to the bacterium cells was verified by visual inspection. The E. coli appeared as yellowish-orange cells, showing that Thioflavin T was bound to the cells and to a lesser extent Congo red. The S. aureus cells were intense red, indicative for Congo red binding. Due to the intense red color, it is possible that yellow Thioflavin T can not be seen. In conclusion, S. aureus binds more Congo red than E. coli, whereas no comparative qualitative measure can be given for Thioflavin T binding. Obviously, Thioflavin T is bound to E. coli.

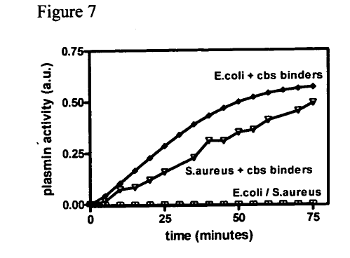

Whether tPA is bound to the E. coli and S. aureus after incubation with 25 μM tPA, was assessed with a tPA/plasminogen chromogenic activation assay, as described above. Plasminogen and chromogenic plasmin substrate Biopep-1751 were mixed with E. coli or S. aureus incubated with buffer only, or with E. coli or S. aureus that were pre-incubated with, amongst other cross-beta structure binding compounds, tPA. Plasmin generation by tPA, measured as conversion of the substrate, can only occur when an external source of tPA activity is introduced in the reaction mixture (FIG. 7).

B. Activation of the Contact System of Blood Coagulation by E. coli with Amyloid Curli Protein

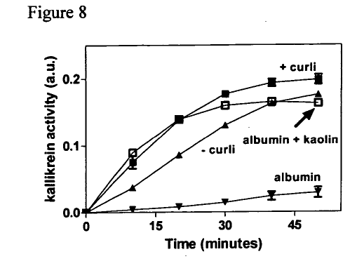

It has been established that E. coli bacteria express an amyloid core protein, curli, at the cell surface, depending on culturing conditions. When E. coli MC4100 is cultured on CFA agar for 44 hours at 26° C., expression of curli is facilitated, whereas no curli is expressed when cells are grown for 24 hours at 37° C. Curli with cross-beta structure has been defined as an important determinant for binding properties of the E. coli towards fibronectin of the host, a cross-beta structure binding protein through the ability of the finger domains (fibronectin type I domains 4, 5, 10, 11 and 12 to bind to proteins comprising cross-beta structure. The E. coli with and without amyloid curli comprising cross-beta structure were applied to a chromogenic factor XII/prekallikrein activation assay. When factor XII becomes activated kallikrein is formed from prekallikrein by activated factor XII, and kallikrein substrate Chromozym-PK is converted, which is measured by absorbance readings in time. In FIG. 8 it is shown that E. coli with curli is a more potent activator of the contact system of coagulation than E. coli lacking curli. Still, E. coli lacking curli is able to activate factor XII. These observations disclose a role for amyloid curli in activating the contact system and it is clear that other activation mechanisms are also present. Perhaps yet undefined alternative cross-beta structure comprising core proteins are exposed on the E. coli. The results make clear that with the in vitro factor XII activation assay differences in amyloid protein load on a pathogen can be depicted.

C4. In Vitro Murine bEnd.3 Endothelial Cell Activation Assay: ROS Production (II)

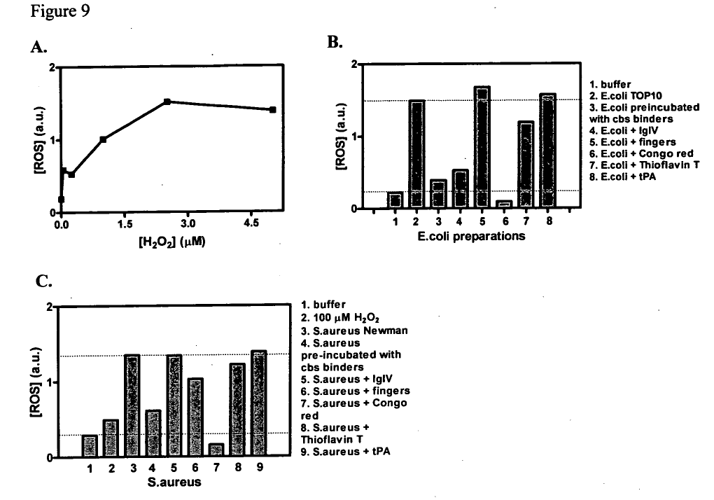

To test whether cross-beta structure binding compounds ThT, Congo red, IgIV, tPA and finger domains of fibronectin, HGFA and tPA have the potency to reverse adverse effects of pathogens E. coli TOP10 and S. aureus Newman on bEnd.3 ECs with respect to ROS expression, the ECs were exposed to 8.1×10⁷ E. coli cells/ml or 1.13×10⁸ S. aureus cells/ml in the presence of buffer, or in the presence of either 1.25 mg/ml IgIV, or 0.8 mg/ml finger domains, or 220 μM Congo red, or 220 μM ThT, or 1.1 μM tPA, and ROS levels were followed in time upon 37° C. incubation. The bacteria were also pre-incubated with ThT, Congo red, IgIV and tPA as described above. From FIG. 9, Panel B, it is clear that pre-incubation of E. coli with cross-beta structure binding compounds inhibits toxic effects of the bacterium towards ECs. Co-incubations of E. coli with IgIV, Congo red and to some extent ThT also reverse ROS production. Finger domains and tPA at the conditions tested are not able to reduce ROS production. For S. aureus, also pre-incubation of the bacterium with Congo red, ThT, tPA and IgIV reduced ROS production by the bEnd.3 cells (FIG. 9, Panel C). In addition, co-incubations of bEnd.3 with S. aureus and Congo red also strongly inhibits ROS expression, whereas finger domains and ThT at the conditions tested slightly decrease ROS production. In contrast to E. coli, in this set-up IgIV does not influence S. aureus induced ROS production. Also tPA does not influence S. aureus induced ROS production in this experimental set-up. Further studies will include refinements of the assay conditions with respect to dosing, experimental settings like buffer, excipients, assay time, cell densities and more. In summary, we conclude that cross-beta structure binding compounds like, for example, ThT, Congo red, finger domains, IgIV are able to reverse adverse effects of pathogens on ECs.

C7. Ex Vivo Human Plasma Coagulation Assays

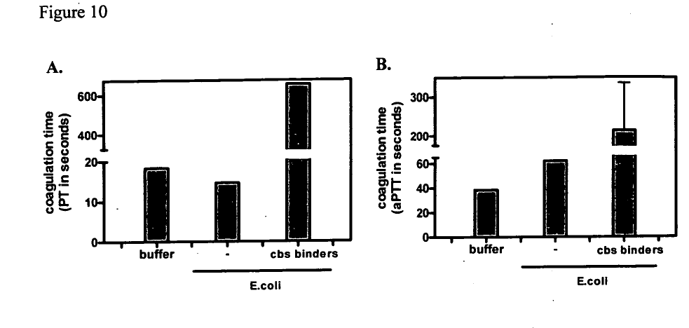

For analysis of the influence of cross-beta structure comprising pathogens on the characteristics of blood coagulation, and for analysis of the effects of cross-beta structure binding compounds on the influence of pathogens on coagulation, aPTT and PT coagulation tests were performed. Pooled human plasma of approximately 40 apparently healthy donors was clotted by adding either negatively charged phospholipids, CaCl₂ and kaolin in the aPTT set-up, or tissue factor rich thromboplastin and CaCl₂ in the PT set-up. Before coagulation tests were performed, two-fold diluted plasma was pre-incubated for approximately one hour at room temperature with PBS (control), 6.5×10⁹ E. coli TOP10 cells/ml, or 6.5×10⁹ E. coli TOP10 cells/ml that were pre-incubated with cross-beta structure binding compounds Congo red, ThT, tPA and IgIV. Before coagulation tests were performed, bacterium cells were pelleted by centrifugation and plasma supernatants were analyzed in the aPTT and PT assays. Results are shown in FIG. 10. The pre-incubation of plasma with E. coli results in acceleration of coagulation with approximately 20% in a PT, and a delayed coagulation with approximately 60% in an aPTT (FIG. 10). Pre-incubations of E. coli with cross-beta structure binding compounds results in a strongly delayed coagulation in both tests. Noteworthy, in several plasma samples that were pre-incubated with bacteria+cross-beta structure binding compounds, initially coagulation was seen, followed by dissolution of the clot (visual inspection). In the PT analyses, measurements last for up to 650 seconds and were stopped when no coagulation was observed at that time. Similarly, aPTT analyses were stopped after approximately 330 seconds when no coagulation had occurred.

From the delayed clotting time in an aPTT with PBS-pre-incubated E. coli control cells we conclude that factors that are essential for the contact system of coagulation are bound to E. coli and subsequently partly depleted from plasma upon pelleting the bacteria. Likely candidates are cross-beta structure binding proteins factor XII and high molecular weight kininogen (see Table 4), and perhaps fibronectin and fibrin. Acceleration of the coagulation time, as seen in PT set-ups after incubation of plasma with E. coli, point to a pro-coagulant activity of the bacterium. Either, cross-beta structure comprising proteins are secreted from the E. coli into the medium, or other pro-coagulant molecules are secreted, or anticoagulant molecules are depleted from plasma upon pelleting the E. coli cells, or (pro)fibrinolytic molecules, like, for example, tPA and plasmin(ogen) are depleted from plasma upon pelleting the E. coli cells, or fibrinolysis inhibitors are secreted into plasma by the pathogen, like, for example, bacterial plasminogen activator inhibitor analogues and/or α2-anti-plasmin analogues, or the plasma is already in a pro-coagulant state upon incubation with E. coli, due to the recruitment of cross-beta structure comprising proteins involved in the contact system of coagulation, i.e., factor XII and HMWK (see Table 4), by cross-beta structure at the E. coli, resulting in activation of the coagulation system. When E. coli are pre-treated with cross-beta structure binding compounds, coagulation is strongly delayed in both PT and aPTT analyses (FIG. 10). This shows that the cross-beta structure binding compounds are indeed bound to the E. coli cells. The strongly delayed coagulation is likely related to bound tPA at the E. coli surface, which can generate plasmin from plasminogen, when a suitable cross-beta structure cofactor is present at the E. coli surface. In the coagulation tests, the plasmin will dissolve fibrin clots that are formed. Apparently, fibrinolytic activity is so strong that a formed clot is again readily lysed or not formed at all.

C3. Maturation of Human Dendritic Cells Induced by Pathogens is Influenced by Cross-Beta Structure Binding Compounds

Immature human DCs were obtained from PBMCs following established protocols. Upon stimulation of the immature DCs for 20 hours with a concentration series of pathogens E. coli TOP10 or S. aureus Newman, which comprise surface cross-beta structure proteins, maturation markers were analyzed with FACS, i.e., down-regulation of endocytic receptors CD36 and CD206 and up regulation of co-stimulatory receptor molecule CD40. The cell surface expression of these markers on control cells that were incubated for 20 hours with medium only, served as a reference for immature DCs. DCs were exposed to 5.1×10⁴−3.25×10⁶ E. coli cells/ml and 7.0×10⁴−4.5×10⁶ S. aureus cells/ml (two-fold dilution series in seven stages). The E. coli and S. aureus were either pre-incubated with PBS, or with cross-beta structure binding compounds ThT, Congo red, tPA and IgIV. At higher pathogen densities, e.g., 1.3×10⁷ E. coli cells/ml or 1.8×10⁷ S. aureus cells/ml, a three- to four-fold increase in FITC background fluorescence was observed when assessing those pathogen samples that were pre-incubated with cross-beta structure binding molecules. Perhaps, ThT excitation and emission wavelengths (435 and 485 nm) are to close to those of FITC (NB FACS contains an argon-ion laser, i.e., 488 nm excitation wavelength; FITC emission channel 530/30 nm, and PE emission channel 675/25 nm), or the signals can be due to auto-fluorescence of bacterium cells. Therefore, the increased background fluorescence is indicative for an interaction of pathogen cells with the DCs. Whether bacterium cells are bound to the DC surface and/or internalized and/or degraded remains to be established. At lower bacterium cell densities, background fluorescence in the FITC and phycoerythrin (PE) channels is not increased in samples comprising pathogens in comparison to non-treated DCs.

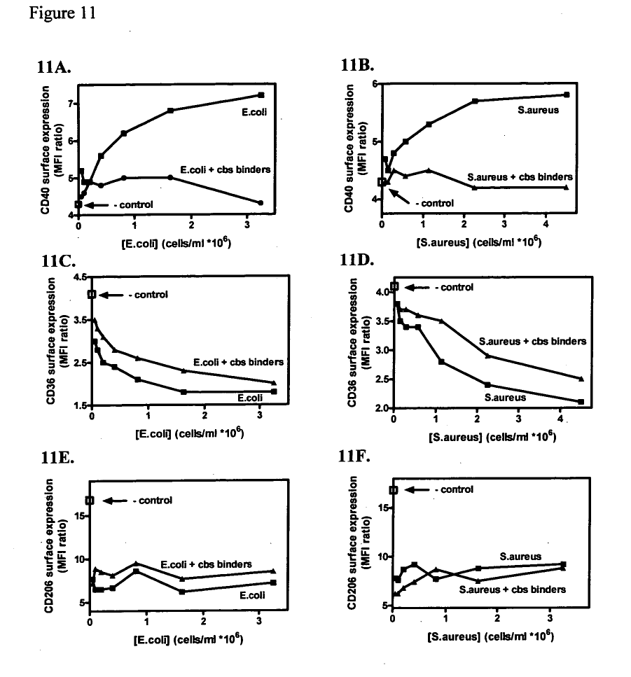

Co-stimulatory receptor CD40 is up-regulated on DCs upon stimulation with all four pathogen preparations, i.e., untreated E. coli, E. coli pre-incubated with cross-beta structure binding compounds, untreated S. aureus and S. aureus pre-incubated with cross-beta structure markers, indicative for maturation of the DCs. The percentage of CD40-positive cells is increased from approximately 60% for the control cells (medium control) that were not stimulated to 70-90% upon stimulation with pathogens (with or without cross-beta structure binding compounds; data not shown). Cell surface expression (MFI ratios) of CD40 increased in a pathogen concentration dependent manner for both E. coli and S. aureus (FIGS. 10A, 11B). However, when the pathogens were pre-incubated with cross-beta structure binding compounds, CD40 surface expression remained similar to that of the untreated non-matured cells (FIGS. 11A, 11B). In conclusion, pre-incubation of the E. coli and S. aureus with ThT, Congo red, tPA and IgIV did not, or hardly, influence the percentage of CD40-positive DCs in comparison to untreated bacteria, though dramatically suppressed the CD40 surface expression on the DCs. Noteworthy, CD40 together with CD40-ligand is one of the known cell surface receptors that interact with amyloid cross-beta structure (Table 4).

Endocytic receptor CD36 is down-regulated on DCs upon stimulation with all four pathogen preparations, i.e., untreated E. coli, E. coli pre-incubated with cross-beta structure binding compounds, untreated S. aureus and S. aureus pre-incubated with cross-beta structure markers, indicative for maturation of the DCs due to exposure to the pathogens. The percentage of CD36-positive cells decreases from approximately 50% with the lowest pathogen cell density, which is comparative to the percentage of CD36-positive cells found for the control DCs (medium control) that were not stimulated, to 5 to 10% upon stimulation with the highest pathogen densities (data not shown). Pre-incubation of pathogens with cross-beta structure binding compounds results in less down-regulation of the number of CD36-positive DCs, compared to buffer-treated pathogen cells, indicative for less potent maturation of the DCs by pathogens that were pre-incubated with cross-beta structure binding compounds ThT, Congo red, tPA and IgIV (data not shown). Cell surface expression (MFI ratios) of CD36 decreased in a pathogen concentration dependent manner for both E. coli and S. aureus (FIGS. 10C, 11D). When the pathogens were pre-incubated with cross-beta structure binding compounds, CD36 surface expression decreased to a lesser extent when compared to the CD36 surface expression on DCs after exposure to buffer-incubated pathogens (FIGS. 11C, 11D). In conclusion, pre-treatment of the E. coli and S. aureus with ThT, Congo red, tPA and IgIV resulted in less down-regulation of CD36, measured as (1) the fraction of CD36-positive cells, and (2) suppressed the decrease in CD36 surface expression on the matured DCs, induced by exposure to the pathogens. These results show that cross-beta structures at the surface of the pathogens most likely actively participate in DC signaling related to maturation. Noteworthy, CD36 is one of the known cell surface multiligand receptors for cross-beta structure that not only interact with pathogens, for example, with S. aureus, but that also interact with amyloid cross-beta structure, like, for example, amyloid-β, glycated proteins and oxidized proteins (Table 4). Therefore, our data are in further support of our proposed “Cross-beta Pathway” for degradation and clearance of obsolete molecules. We propose that an activation mechanism for induction of phagocytic, inflammatory, immunogenic and/or haemostatic activity is triggered by cross-beta structure conformation in proteins. Our current data point to a role for cross-beta structure on both Gram-negative and Gram-positive bacteria in triggering the host immune system, a process in which CD36 is likely actively participating. Interestingly, recently also a second multiligand cross-beta structure receptor has been implicated in the host response to bacteria, i.e., receptor for advanced glycation end-products (RAGE).

The percentage of endocytic receptor CD206-positive DCs is hardly influenced upon stimulation with all four pathogen preparations, i.e., untreated E. coli, E. coli pre-incubated with cross-beta structure binding compounds, untreated S. aureus and S. aureus pre-incubated with cross-beta structure markers, indicating that the mannose receptor does not play an important role in maturation of DCs upon exposure to the pathogens with cross-beta structure surface proteins (data not shown). The percentage of CD206-positive cells stays high at approximately 75 to 90%, whereas non-stimulated cells (medium control) were approximately 90% CD206 positive (data not shown). It seems that lowering the pathogen-induced CD206 cell surface expression results in somewhat decreased number of CD206-positive cells. For all four pathogen preparations endocytic receptor CD206 surface density (MFI ratios) is decreased to a similar extent, indicative for maturation of the DCs upon stimulation with the pathogens (FIGS. 11E, 11F). Because pre-incubation of pathogens with cross-beta structure binding compounds did not influence CD206 surface density on DCs with the pathogen densities tested, we conclude that the mannose receptor CD206 is likely not to be involved in cross-beta structure-mediated signaling.

Analysis of Tissue Factor Expression by THP-1 upon Stimulation with S. aureus that were Pre-Incubated with Buffer or Cross-Beta Structure Binding Compounds

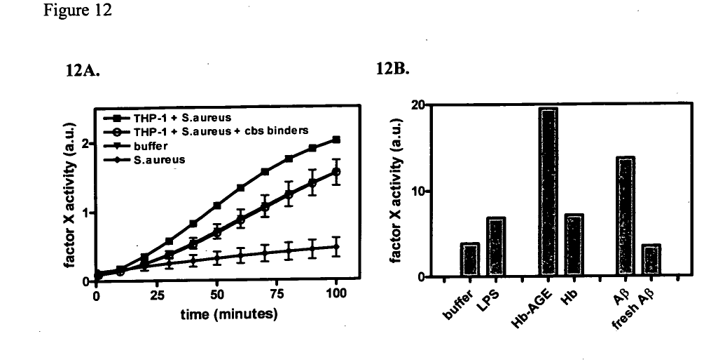

For tissue factor expression analysis purposes, THP-1 cells were exposed to 1.8×10⁷ S. aureus cells/ml for six hours at 37° C. TF activity was determined in an indirect way by assessing activation of factor X in the presence of activated factor VII, with three-fold diluted THP-1 monocyte cell lysate. Factor X activity in THP-1 cell lysates after exposure to PBS-incubated S. aureus was higher than in lysates of cells that were exposed to S. aureus which was pre-incubated with cross-beta structure compounds Thioflavin T, Congo red, tPA and IgIV (FIG. 12A). Pre-incubation of S. aureus with the cross-beta structure binding compounds results in return to basal TF activity levels seen with unstimulated THP-1 cells (FIG. 12A). These results provide a first glimpse on the role of proteins with cross-beta structure at the core of the pathogen in induction of a procoagulant state of the monocytes by TF up-regulation. Cross-beta structure binding compounds effectively inhibit induction of this procoagulant state. Similar experiments with endothelial cells will be conducted to gain insight in whether TF up-regulation is a common aspect of cross-beta structure bearing pathogens, and whether induction of a procoagulant state can be prevented in a broader way.

Misfolded proteins with cross-beta structure conformation, i.e., glycated hemoglobin (Hb-advanced glycation end-product, AGE) and amyloid-β induce elevated levels of TF, as determined by potent factor X activation by THP-1 monocyte lysates after incubation of the cells with the misfolded proteins (FIG. 12B). Freshly dissolved amyloid-β (Aβ) does not induce any additional TF expression. Freshly dissolved hemoglobin (Hb) triggers TF expression similarly as 10 μg/ml lipopolysaccharide (LPS). Perhaps, dissolving lyophilized Hb in TBS does result in partly incorrectly folded protein with cross-beta structure. In further experiments cross-beta structure binding compounds will be tested for their efficient inhibitory activity with respect to induction of a procoagulant state by cross-beta structure. Cross-beta structure binding compounds that potently diminish up-regulation of TF by cross-beta structure can subsequently be tested for their potency to reverse adverse effects of pathogens comprising cross-beta structure on monocytes and ECs.

### SUMMARY

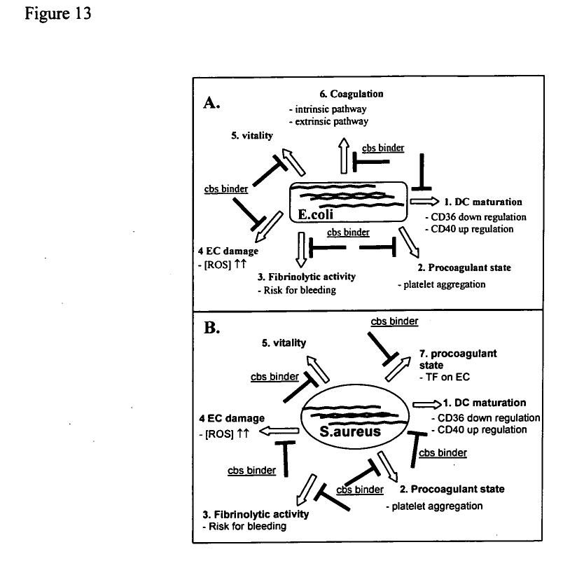

In Table 7 and FIG. 13, all above-discussed data is summarized to give an overview of the various influences of the cross-beta structure on the surface of the pathogens tested in the bioassays.

Abbreviations

APC, antigen-presenting cell; aPTT, activated partial thromboplastin time; BCA, Bicinchoninic Acid; BiP/grp78, Immunoglobulin heavy chain-binding protein/Endoplasmic reticulum lumenal Ca(2+)-binding protein; cbs, cross-beta structure; CD, Cluster of Differentiation; CFA, colonization stimulating factor; DC, dendritic cell; DMEM, Dulbecco's Modified Eagle Medium; EC, endothelial cell; E. coli, Escherichia coli; ELISA, enzyme-linked immunosorbent assay; F, finger domain/fibronectin type I domain; FVII, factor VII; FACS, Fluorescence Activated Cell Sorting; FCS, fetal calf serum; FITC, fluorescein isothiocyanate; Fn, fibronectin; GM-CSF, GRANULOCYTE MACROPHAGE COLONY STIMULATING FACTOR; HBS, HEPES-buffered saline; HEPES, {2-(4-(2-Hydroxyethyl)-1-piperazinyl) ethanesulfonic Acid}; HGFA, hepatocyte growth factor activator; HMWK, high molecular weight kininogen; HSP, heat-shock protein; HLA-DR, D-related human leukocyte antigen; HUVEC, human umbilical vein endothelial cell; IgIV, immunoglobulins intravenous; IMDM, Iscove's Modified Dulbecco's Medium; IL, interleukin; IvIG, intravenous immunoglobulins; IFN, interferon; LB, luria broth; LRP/CD91, low density lipoprotein related receptor; LOX, lecton-like receptor for oxidized low density lipoprotein; MHC, human leukocyte antigen; MTT, mitochondrial metabolic activity; MFI, mean fluorescent intensity; NO, nitric oxide; PBMC, peripheral blood mononuclear cells; PBS, phosphate-buffered saline; PE, phycoerythrin; PRP, platelet rich plasma; PT, prothrombin time; PMA, phorbol 12-myristate 13-acetate; RPMI, Roswell Park Memorial Institute; ROS, reactive oxygen species; S. aureus, Staphylococcus aureus; S. pyogenes, Streptococcus pyogenes; sRAGE, soluble fragment of receptor for advanced glycation end products; TBS, Tris(hydroxymethyl)aminomethane Hydrochloride-buffered saline; ThT, thioflavin T; TLR, Toll-like receptor; TNF-α, tumor necrosis factor-α; TRAP, synthetic thrombin receptor activating peptide; TPA, Tetra-Phorbol-Acetate; tPA, tissue-type plasminogen activator; ULS, universal linkage system.

TABLE 1
Bacterial and fungal species reported
to activate the fibrinolytic system

| Bacterial and fungal | Plasminogen | |
|---|---|---|
| species | activator | Microbial receptors |
| Borellia burgdorferi, | tPA | Outer leaflet cell surface |
| B. coriacae, B. garinii, | | lipoprotein A (OspA), |
| B. anserina, | | borrelial plasminogen |
| B. turicatae, B. hermsii | | binding protein (BPBP) |
| Branhamella | | |
| catarrhalis | | |
| Candida albicans | tPA | phosphoglycerate mutase, |
| | | alcohol dehydrogenase, |
| | | thioredoxin peroxidase, |
| | | catalase, transcription |
| | | elongation factor, |
| | | glyceraldehyde-3-phosphate |
| | | dehydrogenase, phospho- |
| | | glycerate kinase and |
| | | fructose bisphosphate |
| | | aldolase, enolase |
| Escherichia coli | tPA | flagella, Type I, S |
| | | fimbriae, G fimbriae |
| | | (curli) |
| Hemophilus influenza | tPA | Aspartase |
| Helicobacter pylori | tPA | plasminogen binding |
| | | proteins (pgbA and pgbB) |
| Mycobacterium avium, | | |
| N. bovis, | | |
| N. tuberculosis | | |
| Mycoplasma fermans | tPA | |
| Neisseria meningitidus, | tPA | |
| N. gonorrhoeae | | |
| Pneumocystis carinii | | Enolase |
| Proteus mirabilis | tPA | |
| Pseudomonas | | |
| aeruginosa | | |
| Salmonella enterica | tPA | |
| (serovar Typhimurium) | | |
| Staphylococcus aureus | tPA, | |
| | Staphylokinase | |
| Group A, C and G | tPA, | Glyceraldehyde-3- |
| streptococci, | streptokinase | phosphate dehydrogenase |
| Streptococcus | | (GAPDH, SDH, Plr), PAM |
| pneumonia, | | (Plasminogen-binding |
| S. pyogenes, | | group A streptococcal |
| S. equisimilis | | M-like protein), SEN |
| | | (streptococcal surface |
| | | enolase) |
| Streptomyces | tPA | |
| coelicolor | | |
| Treponema denticola | | OppA |
| Yersinia pestis, | Pla | |
| Y. enterocolitica, | | |
| Y pseudotuberculosis | | |

TABLE 2
Bacteria reported to activate the contact system of coagulation

| | | Microbial |
|---|---|---|
| Bacterial species | contact phase proteins | receptors |
| Escherichia coli | Factor XII, high | Curli |
| | molecular weight | |
| | kininogen, prekallikrein | |
| Salmonella enterica | Factor XII, high | Fimbriae |
| (serovar | molecular weight | |
| Typhimurium) | kininogen, prekallikrein | |
| Staphylococcus | — | — |
| aureus | | |
| Streptococcus | — | M proteins |
| pyogenes | | |

TABLE 3
cross-β structure binding compounds

| | | |
|---|---|---|
| Congo red | Chrysamine G | Thioflavin T |
| 2-(4′-(methylamino)phenyl)- | Any other | Glycosaminoglycans |
| 6-methylbenzothiaziole | amyloid-binding | |
| | dye/chemical | |
| Thioflavin S | Styryl dyes | BTA-1 |
| Poly(thiophene acetic acid) | conjugated | |
| | polyeclectrolyte | |
| | PTAA-Li | |

TABLE 4
proteins that bind to and/or interact with cross-β structure
and/or with proteins comprising cross-β structure

| | | |
|---|---|---|
| Tissue-type plasminogen | Finger domain(s) of tPA, | Apolipoprotein E |
| activator | factor XII, fibronectin, HGFA | |
| Factor XII | Plasmin(ogen) | Matrix metalloprotease-1 |
| Fibronectin | 75 kD-neurotrophin receptor | Matrix metalloprotease-2 |
| | (p75NTR) | |
| Hepatocyte growth factor | α2-macroglobulin | Matrix metalloprotease-3 |
| activator | | |
| Serum amyloid P | High molecular weight | Monoclonal antibody |
| component | kininogen | 2C11(F8A6)^{‡} |
| C1q | Cathepsin K | Monoclonal antibody |
| | | 4A6(A7)^{‡} |
| CD36 | Matrix metalloprotease 9 | Monoclonal antibody 2E2(B3)^{‡} |
| Receptor for advanced | Haem oxygenase-1 | Monoclonal antibody 7H1(C6)^{‡} |
| glycation end products | | |
| Scavenger receptor-A | low-density lipoprotein | Monoclonal antibody |
| | receptor-related protein | 7H2(H2)^{‡} |
| | (LRP, CD91) | |
| Scavenger receptor-B | DnaK | Monoclonal antibody 7H9(B9)^{‡} |
| ER chaperone ERp57 | GroEL | Monoclonal antibody 8F2(G7)^{‡} |
| calreticulin | VEGF165 | Monoclonal antibody 4F4^{‡} |
| Monoclonal | Monoclonal conformational | Amyloid oligomer-specific |
| conformational antibody | antibody WO2 (ref. | antibody (ref. (Kayed et al., |
| WO1 (ref. (O'Nuallain and | (O'Nuallain and Wetzel, | 2003)) |
| Wetzel, 2002)) | 2002)) | |
| formyl peptide | α(6)β(1)-integrin | CD47 |
| receptor-like 1 | | |
| Rabbit anti-albumin-AGE | CD40 | apo A-I belonging to small |
| antibody, Aβ-purified^{a)} | | high-density lipoproteins |
| apoJ/clusterin | ten times molar excess | CD40-ligand |
| | PPACK, 10 mM εACA, (100 | |
| | pM - 500 nM) tPA²⁾ | |
| macrophage scavenger | broad spectrum (human) | BiP/grp78 |
| receptor CD163 | immunoglobulin G (IgG) | |
| | antibodies (IgIV, IVIg) | |
| ERdj3 | haptoglobin | |

^{‡}Monoclonal antibodies developed in collaboration with the ABC-Hybridoma Facility, Utrecht University, Utrecht, The Netherlands.
^{a)}Antigen albumin-AGE and ligand Aβ were send in to Davids Biotechnologie (Regensburg, Germany); a rabbit was immunized with albumin-AGE, antibodies against a structural epitope were affinity purified using a column with immobilized Aβ.
²⁾PPACK is Phe-Pro-Arg-chloromethylketone (SEQ-ID 8), εACA is ε-amino caproic acid, tPA is tissue-type plasminogen activator

TABLE 5
Proteins likely to be able to interact with cross-beta structure

| | | |
|---|---|---|
| Monoclonal antibody 4B5 | Heat shock protein 27 | Heat shock protein 40 |
| Monoclonal antibody 3H7^{‡} | Nod2 (=CARD15) | Heat shock protein 70 |
| FEEL-1 | Pentraxin-3 | HDT1 |
| LOX-1 | Serum amyloid A proteins | GroES |
| MD2 | Stabilin-1 | Heat shock protein 90 |
| FEEL-2 | Stabilin-2 | CD36 and LIMPII |
| | | analogous-I (CLA-1) |
| Low Density Lipoprotein | LPS binding protein | CD14 |
| C reactive protein | CD45 | Orosomucoid |
| integrins | alpha-1 antitrypsin | apo A-IV-Transthyretin |
| | | complex |
| albumin | Alpha-1 acid glycoprotein | β2-glycoprotein I |
| Lysozyme | Lactoferrin | Megalin |
| Tamm-Horsfall protein | Apolipoprotein E3 | Apolipoprotein E4 |
| Toll-like receptors | Complement receptor | CD11d/CD18 (subunit aD) |
| | CD11b/CD18 (Mac-1, CR3) | |
| CD11b2 | CD11a/CD18 (LFA-1, subunit | CD11c/CD18 (CR4, subunit |
| | aL) | aX) |
| Von Willebrand factor | Myosin | Agrin |
| Perlecan | Hsp60 | b2 integrin subunit |
| proteins that act in the | proteins that act in the | Macrophage receptor with |
| unfolded protein response | endoplasmic reticulum stress | collagenous structure |
| (UPR) pathway of the | response (ESR) pathway of | (MARCO) |
| endoplasmic reticulum (ER) | prokaryotic and eukaryotic cells | |
| of prokaryotic and | | |
| eukaryotic cells | | |
| 20S | HSP16 family members | HSC73 |
| HSC70 | translocation channel protein | 26S proteasome |
| | Sec61p | |
| 19S cap of the proteasome | UDP-glucose: glycoprotein | carboxy-terminus of |
| (PA700) | glucosyl transferase (UGGT) | HSP70-interacting protein |
| | | (CHIP) |
| Pattern Recognition | Derlin-1 | Calnexin |
| Receptors | | |
| Bcl-2 asociated athanogene | GRP94 | Endoplasmic reticulum p72 |
| (Bag-1) | | |
| (broad spectrum) (human) | proteins that act in the | The (very) low density |
| immunoglobulin M (IgM) | endoplasmic reticulum | lipoprotein receptor family |
| antibodies | associated degradation system | |
| | (ERAD) | |
| Fc receptor | | |

^{‡}Monoclonal antibodies developed in collaboration with the ABC-Hybridoma Facility, Utrecht University, Utrecht, The Netherlands.

TABLE 6
Pathogens and their associated pathology

| Pathogen | Pathological condition |
|---|---|
| Borellia burgdorferi | Lyme disease |
| Candida albicans | Candidiasis, thrush |
| Escherichia coli | Gastrointestinal disease |
| Hemophilus influenza | Infectious meningitis |
| Helicobacter pylori | Gastritis, ulcers |
| Mycobacterium avium | Tuberculosis |
| M.. tuberculosis | Tuberculosis |
| Neisseria meningitidus | Meningitis |
| N. gonorrhoeae | gonorrhea |
| Pneumocystis carinii | pneumonia |
| Proteus mirabilis | Urinary tract infection |
| Pseudomonas aeruginosa | Infections of lungs and skin etc |
| Salmonella enterica (serovar | Gastrointestinal disease |
| Typhimurium) | |
| Staphylococcus aureus | Purulent discharges, mastitis, |
| | endocarditis |
| Streptococcus pneumonia | pneumonia |
| Streptococcus pyogenes | Scarlet fever, rheumatic fever, strep |
| | throat |
| Yersinia pestis | plaque |
| Y. enterocolitica | Enteric fever |

References:
http://www.ncbi.nlm.nih.gov/entrez/query.fcgi?CMD=search&DB=pubmed and Madigan M T, Martinko J M, Parker J, Brock Biology of Microorganisms, Prentice Hall, 10^{th} edition, ISBN 0-13-049147-0.

TABLE 7
Summary of effects of cross-beta structure binding compounds on the
bioactivity of pathogens E. coli and S. aureus

| | Pathogen¹⁾ | | | | | |
|---|---|---|---|---|---|---|
| | | | | E. coli | | |
| | E. coli | E. coli | | TOP10 + | | S. aureus + |
| | MC4100 + | MC4100 − | E. coli | cbs | S. aureus | cbs |
| (bio)activity | curli | curli | TOP10 | binders | Newman | binders |
| Vitality^{‡} | | | ++ | − | ++ | + (˜40%) |
| tPA binding | | | + | | + | |
| ThT binding | | | + | | | |
| Congo red binding | | | +/− | | ++ | |
| ROS expression by | | | + | +/− to − | + | +/− to − |
| bEnd.3 ECs | | | | | | |
| Factor XII activation | + | +/− | + | − | | |
| Coagulation (PT) | | | accelerated | delayed | Slightly | delayed |
| | | | | | delayed | |
| Coagulation (aPTT) | | | delayed | Strongly | | |
| | | | | delayed | | |
| Platelet aggregation | | | + | +/− | ++ | + |
| DC maturation | | | | | | |
| markers | | | | | | |
| CD36 | | | + | +/− | + | +/− |
| down-regulation | | | | | | |
| CD206 | | | ++ | ++ | ++ | ++ |
| down-regulation | | | | | | |
| CD40 up-regulation | | | + | − | + | − |
| Tissue factor | | | | | ++ | + |
| expression by THP-1 | | | | | | |
| monocytes | | | | | | |

^{‡}Vitality of the bacteria was determined after treatment with buffer or with cross-beta structure binding compounds.
¹⁾“cbs binders” refers to pre-incubation of pathogen with cross-beta structure binding compounds, before applying the pathogen in the assays.

### REFERENCES

1. Bouma B., L. M. Kroon-Batenburg, Y. P. Wu, B. Brunjes, G. Posthuma, O. Kranenburg, P. G. de Groot, E. E. Voest, and M. F. Gebbink (2003). Glycation induces formation of amyloid cross-beta structure in albumin. J. Biol. Chem. 278:41810-41819.

2. Citterio S., M. Rescigno, M. Foti, F. Granucci, D. Aggujaro, C. Gasperi, M. K. Matyszak, G. Girolomoni, and P. Ricciardi-Castagnoli (1999). Dendritic cells as natural adjuvants. Methods 19:142-147.

3. Claessen D., R. Rink, W. de Jong, J. Siebring, P. de Vreugd, F. G. Boersma, L. Dijkhuizen, and H. A. Wösten (2003). A novel class of secreted hydrophobic proteins is involved in aerial hyphae formation in Streptomyces coelicolor by forming amyloid-like fibrils. Genes Dev. 17:1714-1726.

4. Gebbink M. F., D. Claessen, B. Bouma, L. Dijkhuizen, and H. A. Wösten (2005). Amyloids—a functional coat for microorganisms. Nat. Rev. Microbiol. 3:333-341.

5. Kranenburg O., B. Bouma, L. M. Kroon-Batenburg, A. Reijerkerk, Y. P. Wu, E. E. Voest, and M. F. Gebbink (2002). Tissue-type plasminogen activator is a multiligand cross-beta structure receptor. Curr. Biol. 12:1833-1839.

6. Sallusto F. and A. Lanzavecchia (1994). Efficient presentation of soluble antigen by cultured human dendritic cells is maintained by granulocyte/macrophage colony-stimulating factor plus interleukin 4 and down-regulated by tumor necrosis factor alpha. J. Exp. Med. 179:1109-1118.

1. A method of treating a microbial infection in a subject, the method comprising:
administering a protease inhibitor to the subject so as to treat the microbial infection.

2. A method of treating a microbial infection in a subject, the method comprising:
administering a medicament to the subject, the medicament comprising:
means for binding to a cross-β structure, and
a pharmaceutically acceptable excipient,
so as to treat the microbial infection in the subject.

3. The method according to claim 2, wherein said means for binding to a cross-β structure comprises a composition selected from the group consisting of an antibody, an antibody fragment, and a finger domain of a protease, said finger domain specific for a cross-β structure.

4. The method according to claim 3, wherein said composition is a bi-specific antibody against a cross-β structure and a microbial antigen.

5. The method according to claim 2, wherein said microbial infection is caused by a microorganism selected from the group consisting of a pathogenic microorganism, gram-positive bacteria, fungus, actinomycete bacteria, streptomycete bacteria, gram-negative bacteria, E. coli, and Salmonella.

6. The method according to claim 2, wherein a cross-β structure comprises a hydrophobin or a chaplin.

7. The method according to claim 1, wherein said microbial infection is caused by an organism selected from the group consisting of fungus, gram-positive bacteria, actinomycete, and streptomycete.

8. The method according to claim 2, wherein a cross-β structure comprises a curli, a Tafi, or a thin aggregative fimbria.

9. The method according to claim 2, wherein said microbial infection is caused by a bacteria selected from the group consisting of gram-negative bacteria, E. coli, and Salmonella.

10. A method for producing a less virulent microorganism, said method comprising:
deleting at least a part of a gene of said microorganism encoding an amyloid fibril forming protein,
so as to produce a less virulent form of said microorganism.

11. An immunogenic composition comprising a microorganism from which at least one gene, encoding a cross-β structure forming protein, has been deleted.

12. A composition comprising a protease inhibitor and a fungicide.

13. The composition of claim 12, wherein the protease inhibitor is a serine protease inhibitor.

14. A composition comprising a protease inhibitor and a bactericide.

15. The composition of claim 14, wherein the protease inhibitor is a serine protease inhibitor.

16. A composition comprising a protease inhibitor and a cross-β structure-binding compound.

17. The composition of claim 16, wherein said cross-β structure-binding compound comprises a composition selected from the group consisting of an antibody, an antibody fragment, and a finger domain of a protease, said finger domain specific for a cross-β structure.

18. The composition of claim 17, further comprising a bactericide or a fungicide.

19. A kit for detecting microbial contamination of a solution and/or a substance, said kit comprising:
a cross-β structure binding compound and
means for detecting binding of said cross-β structure to said binding compound.

20. The kit of claim 19, wherein the means for detecting binding of a cross-β structure to the binding compound comprises:
a tPA- and/or factor XII activation assay, or
visualization of a staining compound.
